(19) United States
(12) Patent Application Publication
(10) Pub. No.: US 2025/0345479 A1
(43) Pub. Date: Nov. 13, 2025

(54) PH-SENSITIVE WOUND DRESSING

(71) Applicant: Elizabeth Li-Eng Chong, Dallas, TX (US)

(72) Inventor: Elizabeth Li-Eng Chong, Dallas, TX (US)

(21) Appl. No.: 19/201,111

(22) Filed: May 7, 2025

Related U.S. Application Data
(60) Provisional application No. 63/645,323, filed on May 10, 2024.

(51) Int. Cl. A61L 26/00; A61B 5/00; A61B 5/145; C08L 89/06

(52) U.S. Cl. CPC A61L 26/008; A61B 5/14539; A61B 5/6802; A61L 26/0038; A61L 26/0066; C08L 89/06; A61L 2300/404

(57) ABSTRACT

A system and method are disclosed for monitoring a wound for infection with a pH-sensitive wound dressing. In one aspect, a method for monitoring a wound for infection involves the steps of positioning a pH-sensitive wound dressing in direct contact with the wound, maintaining contact between the pH-sensitive wound dressing and the wound for an observation period, and evaluating whether the pH-sensitive wound dressing reflects a colorimetric change during the observation period. The pH-sensitive wound dressing includes a hydrogel configured for contact with the wound, a pH-indicating dye that is incorporated within the hydrogel, and a hydrogel covering configured to maintain contact between the hydrogel and the wound. The pH-indicating dye demonstrates the colorimetric change within a pre-determined pH range.

### RELATED APPLICATIONS

This application claims the benefit of U.S. Patent Application No. 63/645,323 filed May 10, 2024, and entitled “PH-Sensitive Wound Dressing for Early Detection of Infection in All Skin Color Types,” the disclosure of which is herein incorporated by reference.

### FIELD OF THE INVENTION

Embodiments herein generally relate to medical wound monitoring and, more particularly, to pH-sensitive wound dressings, methods of making the pH-sensitive wound dressings, and methods of monitoring for wound infection using the pH-sensitive wound dressings.

### BACKGROUND OF THE INVENTION

Surgical site infections (SSIs) are the most prevalent hospital-acquired infections. SSIs and chronic wound infections create significant suffering for patients and burdens on the healthcare system. There are approximately 160,000 to 300,000 SSIs each year in the United States. SSIs account for 20% of hospital-acquired infections and occur in 2% to 5% of patients undergoing surgery. SSIs are also the most expensive hospital-acquired infections, costing from $3.5 billion to $10 billion each year in the United States. Furthermore, SSIs increase a patient's average time in the hospital by about 9.7 days and can bring about long-lasting impairments. Severe non-healing infected wounds can lead to bacterial spread into the blood stream and then other body parts which can lead to end organ damage, amputation, and even death. Additionally, SSIs heighten the patient's risk of death (11-fold compared to patients without SSIs), and roughly 75% of the deaths for patients that contract an SSI are caused by the SSI.

The cause of an SSI may be extrinsic or intrinsic. Extrinsic causes include unsanitary instruments, contaminated surgical setting, or lack of proper hand hygiene by a healthcare provider. Intrinsic causes include bacterial contamination from the patient's own skin, gastrointestinal tract, mouth, or eye. The risk of contracting an SSI heightens with longer surgeries, greater blood loss, specific types of anesthesia, and participation of two or more residents (trainees) in the patient's surgery.

Early identification of SSIs and other types of wound infections (such as traumatic or dependent decubitis ulcers, i.e., bed sores) can greatly increase chance of resolution. The most common method for identifying wound infections is to monitor clinically for symptoms or signs of an infection such as worsening pain, redness, swelling, and pus formation. Unfortunately, these signs and symptoms usually do not present until later stages of infection. The recurring limitation to early identification of wound infection is that the patient, doctor, or other medical practitioner must see, feel, smell, or touch the patient to identify symptoms or signs of infection. A patient must typically travel to a healthcare facility for a medical professional to make the clear diagnosis of an infection.

Several elegant devices have been developed for identifying early infection in wounds. These devices include smartphone programs, wearable devices, and flexible electronics. Such devices, however, have limited practical utility due to their high cost, complexity of readout, and lack of availability to patients, particularly in poorer socioeconomic settings or in developing countries.

It is therefore desirable to implement improved systems and methods to detect wound infection prior to the manifestation of signs or symptoms, which may allow for earlier treatment and resolution of the infection to improve quality of life and mortality. It is also desirable to implement systems and methods to detect wound infection that use a simple, visual readout and do not rely on specialized equipment.

Before proceeding to a detailed description of the invention, it should be noted and remembered that the description of the invention which follows, together with the accompanying drawings, should not be construed as limiting the invention to the examples (or embodiments) shown and described. Those skilled in the art to which the invention pertains will be able to devise other forms of this invention within the ambit of the appended claims.

### SUMMARY OF THE INVENTION

The invention generally relates to systems and methods for monitoring a medical wound for infection.

In one aspect, a pH-sensitive wound dressing includes a hydrogel configured for contact with a wound, a pH-indicating dye that is incorporated within the hydrogel, and a hydrogel covering configured to maintain contact between the hydrogel and the wound. The pH-indicating dye demonstrates a colorimetric change within a pre-determined pH-range. In embodiments, such color change may be indicative of an infected state at the wound site.

In an embodiment, the hydrogel includes a natural polymer, a synthetic polymer, or a combination thereof.

In an embodiment, the natural polymer is gelatin, collagen, agar-agar, hyaluronic acid, chitosan, heparin, alginate, fibrin, bacterial nanocellulose, or a combination or copolymer thereof.

In an embodiment, the synthetic polymer is polyvinyl alcohol, polyethylene glycol, sodium polyacrylate, polyvinylpyrrolidone, or a combination or copolymer thereof.

In an embodiment, the hydrogel also includes an anti-microbial agent, a dye-retention additive, or both.

In an embodiment, the hydrogel is visually divided into sections to aid identification of a nexus of infection for the wound.

In an embodiment, the pre-determined pH range of the pH-indicating dye is between about 4 and about 9.

In an embodiment, the pH-indicating dye is bromothymol blue, azolitmin, neutral red, methylene blue, or a combination thereof.

In an embodiment, the hydrogel comprises a natural polymer, more particularly gelatin, and the pH-indicating dye is bromothymol blue.

In an embodiment, the pH-indicating dye is configured to exhibit a first colorimetric change from yellow to blue, followed by a second colorimetric change from blue to yellow.

In another aspect, a method for monitoring a wound for infection involves positioning a pH-sensitive wound dressing in direct contact with the wound, maintaining contact between the pH-sensitive wound dressing and the wound for an observation period, and evaluating whether the pH-sensitive wound dressing reflects a colorimetric change during the observation period. The pH-sensitive wound dressing includes a hydrogel that is configured to contact the wound, a pH-indicating dye that is incorporated within the hydrogel, and a hydrogel covering that is configured to maintain contact between the hydrogel and the wound. The pH-indicating dye demonstrates the colorimetric change within a pre-determined pH range. In embodiments, such color change may be indicative of an infected state at the wound site.

In an embodiment, the observation period ranges from one second to 100 hours after the initial contact of the wound with the pH-sensitive wound dressing.

In an embodiment, the step of evaluating whether the pH-sensitive wound dressing reflects a colorimetric change comprises the step of checking the pH-sensitive wound dressing for the colorimetric change at a pre-determined observation interval.

In an embodiment, the step of evaluating whether the pH-sensitive wound dressing reflects a colorimetric change is performed by visual inspection or using a colorimeter.

In an embodiment, the step of evaluating whether the pH-sensitive wound dressing reflects a colorimetric change is performed by comparing the color of the hydrogel to a reference color scale by visual inspection, where the reference color scale depicts colors that are expected from the pH-indicating dye for pre-specified pH values within the pre-determined pH-range.

In an embodiment, the method for monitoring the wound for infection also includes the step of observing the pH-sensitive wound dressing for a second colorimetric change after the colorimetric change is detected.

In yet another aspect, a method for preparing a pH-sensitive wound dressing includes the steps of adsorbing a pH-indicating dye within a hydrogel, positioning the hydrogel in direct contract with a wound, and placing a hydrogel covering over the hydrogel. The pH-indicating dye demonstrates a colorimetric change within a pre-determined pH range, and the hydrogel covering is configured to maintain contact between the hydrogel and the wound.

In an embodiment, the step of adsorbing the pH-indicating dye involves the steps of mixing the pH-indicating dye with a hydrogel solution and allowing the hydrogel solution to solidify to form the hydrogel.

In an embodiment, the step of adsorbing the pH-indicating dye involves the steps of allowing a hydrogel solution to solidify to form the hydrogel, covering the hydrogel with a layer of the pH-indicating dye, and permitting the hydrogel to adsorb the pH-indicating dye for a predetermined adsorption period.

In an embodiment, the step of allowing the hydrogel solution to solidify involves incubating the hydrogel solution at room temperature.

### BRIEF DESCRIPTION OF DRAWINGS

The above and other objects and advantages of this invention may be more clearly seen when viewed in conjunction with the accompanying drawing wherein:

FIG. 1A depicts a perspective view of a pH-sensitive wound dressing constructed in accordance with an exemplary embodiment.

FIG. 1B depicts an exploded perspective view of a pH-sensitive wound dressing constructed in accordance with an exemplary embodiment.

FIG. 2A depicts B-values of hydrogels made with gelatin and collagen with 0.1% bromothymol blue (BTB) added post-solidification of the hydrogel after 10 hours in different pHs.

FIG. 2B depicts B-values of hydrogels made with gelatin and collagen with 0.1% bromothymol blue (BTB) added post-solidification of the hydrogel after 1 hour in different pHs.

FIG. 2C depicts B-values of hydrogels made with gelatin and collagen with 0.1% bromothymol blue (BTB) added before solidification of the hydrogel after 10 hours in different pHs.

FIG. 2D depicts B-values of hydrogels made with gelatin and collagen with 0.1% bromothymol blue (BTB) added before solidification of the hydrogel after 1 hour in different pHs.

FIG. 3 depicts the layers of a pH-sensitive wound dressing constructed in accordance with an exemplary embodiment and a simulated wound bed model.

FIG. 4 depicts B-values (median, n=9) after 10 hours of hydrogels made with 5 grams of gelatin in 75 mL of distilled water and 5 drops of 0.1% BTB added after solidification.

FIG. 5 depicts a series of 0.67% gelatin hydrogels with 0.25 mg of BTB after 10 hours in pH 5.0, pH 5.75, pH 6.5, pH 7.25, and pH 8.0 against different Fitzpatrick skin color backgrounds.

FIG. 6A depicts B-values of the hydrogels of FIG. 4 after 10 hours in pH 5.0 compared to pH 5.75, pH 6.5, pH 7.25, and pH 8.0 on a white background.

FIG. 6B depicts B-values of the hydrogels of FIG. 4 after 10 hours in pH 5.0 compared to pH 5.75, pH 6.5, pH 7.25, and pH 8.0 on Type 1 Fitzpatrick skin color.

FIG. 6C depicts B-values of the hydrogels of FIG. 4 after 10 hours in pH 5.0 compared to pH 5.75, pH 6.5, pH 7.25, and pH 8.0 on Type 2 Fitzpatrick skin color.

FIG. 6D depicts B-values of the hydrogels of FIG. 4 after 10 hours in pH 5.0 compared to pH 5.75, pH 6.5, pH 7.25, and pH 8.0 on Type 3 Fitzpatrick skin color.

FIG. 6E depicts B-values of the hydrogels of FIG. 4 after 10 hours in pH 5.0 compared to pH 5.75, pH 6.5, pH 7.25, and pH 8.0 on Type 4 Fitzpatrick skin color.

FIG. 6F depicts B-values of the hydrogels of FIG. 4 after 10 hours in pH 5.0 compared to pH 5.75, pH 6.5, pH 7.25, and pH 8.0 on Type 5 Fitzpatrick skin color.

FIG. 6G depicts B-values of the hydrogels of FIG. 4 after 10 hours in pH 5.0 compared to pH 5.75, pH 6.5, pH 7.25, and pH 8.0 on a Type 6 Fitzpatrick skin color background.

FIG. 7 depicts B-values of the hydrogel of FIG. 4 in accordance with an exemplary embodiment on a white background over time at different pHs.

FIG. 8A depicts median B-values (n=9) with interquartile range (IQR) of the left (pH 5.0) and right side (pH 5.0, pH 5.75, pH 6.5, pH 7.25, pH 8.0) of hydrogels on Type 1 Fitzpatrick skin color.

FIG. 8B depicts median B-values (n=9) with IQR of the left (pH 5.0) and right side (pH 5.0, pH 5.75, pH 6.5, pH 7.25, pH 8.0) of hydrogels on Type 6 Fitzpatrick skin color.

FIG. 9A depicts hydrogels on pieces of gauze made up of ¾ dipped in pH 5.0 solution, ¼ dipped in varying pH (pH 5.0, pH 5.75, pH 6.5, pH 7.25, pH 8.0).

FIG. 9B depicts hydrogels on pieces of gauze made up of ⅞ dipped in pH 5.0 solution, ⅛ dipped in varying pH (pH 5.0, pH 5.75, pH 6.5, pH 7.25, pH 8.0).

FIG. 9C depicts hydrogels on pieces of gauze made up of 15/16 dipped in pH 5.0 solution, 1/16 dipped in varying pH (pH 5.0, pH 5.75, pH 6.5, pH 7.25, pH 8.0).

FIG. 10A depicts a flow diagram of the experimental designs for Examples 6 and 7, where hydrogels were placed on top of pH 5.0 gauze and were incubated for various time points (5 hours, 10 hours, 24 hours, or 48 hours) before being transferred to a gauze of a designated pH (pH 5.0, pH 5.75, pH 6.5, pH 7.25, or pH 8.0) (“Switch 1”), then transferred again to a new pH 5.0 gauze (“Switch 2”), with color measurements performed before and immediately after Switch 1, before Switch 2, and at 10 hours after Switch 2.

FIG. 10B depicts median B-value (n=9) with IQR of hydrogels placed on pH 5.0 simulated wound bed (for 5 hours, 10 hours, 24 hours, or 48 hours) then transferred to pH 5.0 simulated wound bed for 10 hours then transferred back to pH 5.0 simulated wound bed, all on Type 1 Fitzpatrick skin color.

FIG. 10C depicts median B-value (n=9) with IQR of hydrogels placed on pH 5.0 simulated wound bed (for 5 hours, 10 hours, 24 hours, or 48 hours) then transferred to pH 5.0 simulated wound bed for 10 hours then transferred back to pH 5.0 simulated wound bed, all on Type 6 Fitzpatrick skin color.

FIG. 10D depicts median B-value (n=9) with IQR of hydrogels placed on pH 5.0 simulated wound bed (for 5 hours, 10 hours, 24 hours, or 48 hours) then transferred to pH 5.75 simulated wound bed for 10 hours then transferred back to pH 5.0 simulated wound bed, all on Type 1 Fitzpatrick skin color.

FIG. 10E depicts median B-value (n=9) with IQR of hydrogels placed on pH 5.0 simulated wound bed (for 5 hours, 10 hours, 24 hours, or 48 hours) then transferred to pH 5.75 simulated wound bed for 10 hours then transferred back to pH 5.0 simulated wound bed, all on Type 6 Fitzpatrick skin color.

FIG. 10F depicts median B-value (n=9) with IQR of hydrogels placed on pH 5.0 simulated wound bed (for 5 hours, 10 hours, 24 hours, or 48 hours) then transferred to pH 6.5 simulated wound bed for 10 hours then transferred back to pH 5.0 simulated wound bed, all on Type 1 Fitzpatrick skin color.

FIG. 10G depicts median B-value (n=9) with IQR of hydrogels placed on pH 5.0 simulated wound bed (for 5 hours, 10 hours, 24 hours, or 48 hours) then transferred to pH 6.5 simulated wound bed for 10 hours then transferred back to pH 5.0 simulated wound bed, all on Type 6 Fitzpatrick skin color.

FIG. 10H depicts median B-value (n=9) with IQR of hydrogels placed on pH 5.0 simulated wound bed (for 5 hours, 10 hours, 24 hours, or 48 hours) then transferred to pH 7.25 simulated wound bed for 10 hours then transferred back to pH 5.0 simulated wound bed, all on Type 1 Fitzpatrick skin color.

FIG. 10I depicts median B-value (n=9) with IQR of hydrogels placed on pH 5.0 simulated wound bed (for 5 hours, 10 hours, 24 hours, or 48 hours) then transferred to pH 7.25 simulated wound bed for 10 hours then transferred back to pH 5.0 simulated wound bed, all on Type 6 Fitzpatrick skin color.

FIG. 10J depicts median B-value (n=9) with IQR of hydrogels placed on pH 5.0 simulated wound bed (for 5 hours, 10 hours, 24 hours, or 48 hours) then transferred to pH 8.0 simulated wound bed for 10 hours then transferred back to pH 5.0 simulated wound bed, all on Type 1 Fitzpatrick skin color.

FIG. 10K depicts median B-value (n=9) with IQR of hydrogels placed on pH 5.0 simulated wound bed (for 5 hours, 10 hours, 24 hours, or 48 hours) then transferred to pH 8.0 simulated wound bed for 10 hours then transferred back to pH 5.0 simulated wound bed, all on Type 6 Fitzpatrick skin color.

FIG. 11 depicts median B-value (n=8) with IQR of hydrogel placed on gauze soaked in solution (pH 5.0, pH 5.75, pH 6.5, pH 7.25, pH 8.0) on ex vivo porcine skin samples.

### DETAILED DESCRIPTION

While this invention is susceptible to embodiment in many different forms, there are shown in the drawings and will herein be described hereinafter in detail some specific embodiments of the invention. It should be understood, however, that the present disclosure is to be considered an exemplification of the principles of the invention and is not intended to limit the invention to the specific embodiments so described.

As a first matter, the pH of a skin wound (expressed as the negative logarithm of H+ concentration, pH=−log [H⁺]) has been identified as a major indicator of wound infection. Although many variables such as age, ethnicity, sebum, sweat, soaps, and cosmetic products can affect skin pH, the pH value of healthy skin is relatively consistent for all humans. The acid mantle makes up the outermost layer of the epidermis and keeps skin at an acidic pH value of 4.2 to 5.6 in healthy adults and children. Skin heals best in an acidic environment, which promotes the formation of regenerative tissue and manages microbial levels.

In contrast, infected wounds typically have an alkaline pH of 7.0 to 9.0. Alkalinity is not desirable for healing skin wounds, as alkaline pH promotes both bacterial growth and infection and otherwise reduces skin healing. For example, alkaline pH may prevent the progression of the four normal stages of healing: hemostasis, inflammation, proliferation, and remodeling. At an alkaline pH of 8.0, enzymes that break down tissue (such as matrix metalloproteinases (MMPs), elastase, and plasmin) also become more active. MMPs are a group of over 20 proteases (i.e., enzymes that break down proteins to amino acids) that function best in an environment with pH>6. MMPs are used in many body processes, including wound healing and embryogenesis. Tissue inhibitors of MMPs (TIMPs) counteract MMPs, and this balance of MMPs and TIMPs is essential for skin healing. In chronic wounds, an alkaline pH drives MMP overactivity which can cause the proteases to additionally degrade new tissue, inhibiting the healing process and leaving open wounds, which may lead to further infection.

In terms of promoting bacterial growth and infection, alkaline pH may signal the presence of bacteria. Ammonia is released by metabolic reactions in bacteria, which raises pH. Alkaline pH of a wound has been associated with bacterial infection and, in local infections, pH increases may occur before any clinical symptoms of the infection are detectable. Measurement of wound pH may therefore be used as a diagnostic and therapeutic indicator of infection in wound healing.

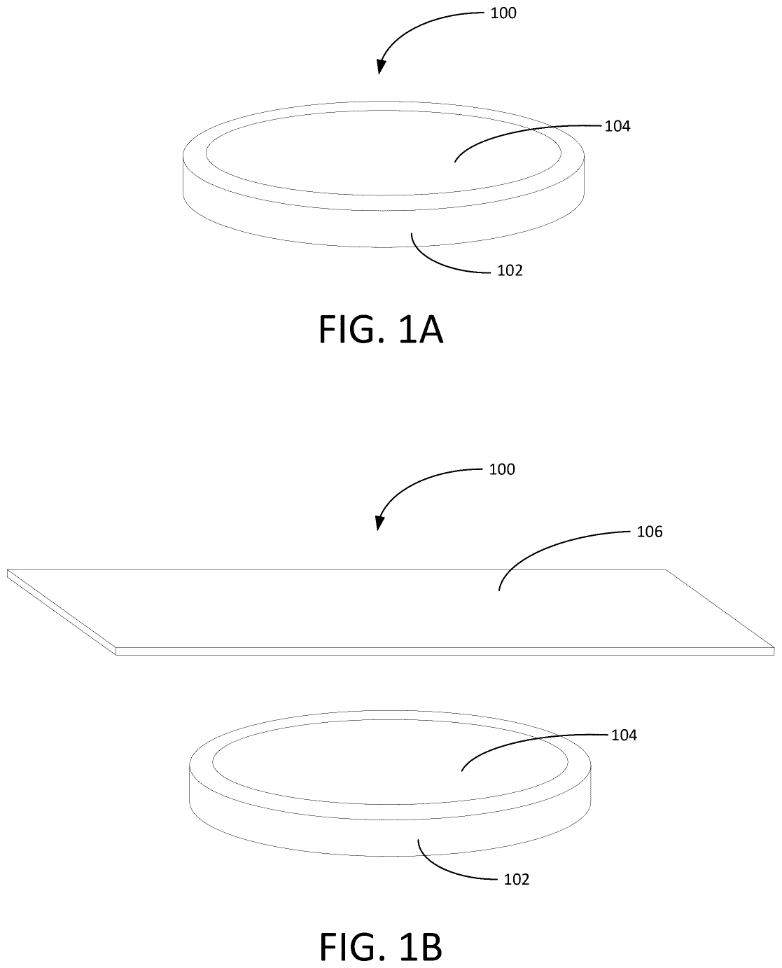

Turning to the perspective views of FIGS. 1A and 1B, a pH-sensitive wound dressing 100 is disclosed for monitoring a wound for infection. The pH-sensitive wound dressing 100 includes a hydrogel 102 that is configured for direct contact with the wound, a pH-indicating dye 104 that is incorporated within the hydrogel 102 and, optionally, a hydrogel covering 106 that is configured to maintain contact between the hydrogel 102 (incorporating the pH-indicating dye 104) and the wound. In certain embodiments that include the hydrogel covering 106, the hydrogel covering 106 is incorporated with the hydrogel 102 and the pH-indicating dye 104 as a single unit. As such, the hydrogel covering 106 and the hydrogel 102 having the pH-indicating dye 104 are positioned as a single unit against a wound. In other embodiments that include the hydrogel covering 106, the hydrogel covering 106 is a separate component from the hydrogel 102 and the pH-indicating dye 104 and is placed over the hydrogel 102 having the pH-indicating dye 104 after the hydrogel 102 is positioned against a wound. It will be appreciated that, in other embodiments, the pH-sensitive wound dressing 100 includes the hydrogel 102 having the pH-indicating dye 104 but does not include the hydrogel covering 106. As a non-limiting example, such embodiments of the pH-sensitive wound dressing 100 may be useful in settings where the patient or medical professional wishes to place the hydrogel 106 against a wound, obtain a readout within a short time frame (ranging from, e.g., 1 second to 5 minutes), and then immediately remove the hydrogel 106 from against the wound.

The pH-sensitive wound dressing 100 provides a visual colorimetric readout. More particularly, the pH-sensitive wound dressing 100 creates a distinct color change as the pH-indicating dye 104 responds to changes in the wound's pH by changing color. This color change is easy to visually interpret, e.g., by the unaided eye or through use of a colorimeter, color strips, or some other color-detecting equipment. As the pH of the wound shifts in response to the onset of an infection (i.e., becomes more alkaline), the pH-sensitive wound dressing 100 signals the shift with a distinct color change. The colorimetric change of the pH-indicating dye 104 produces sufficient contrast for the pH-sensitive wound dressing 100 to be read out against a wide range of skin colors (e.g., Types 1-6 on the Fitzpatrick scale). In various embodiments, the pH-sensitive wound dressing 100 is analyzed by the wearer or a health care provider without removing the wound dressing 100 from the wound, as removing the pH-sensitive wound dressing 100 might peel off healing tissue or introduce new bacteria.

It will be appreciated that the term “hydrogel,” as used herein, refers to a gel in which water is used as a swelling agent. Although the hydrogels 102 of FIGS. 1A and 1B are depicted as a circular disc, it will be appreciated that the hydrogel 102 may be otherwise shaped to cover the wound (e.g., square, rectangular, triangular, oval, irregular). In an exemplary embodiment, the hydrogel 102 is shaped as a disc with a thickness of between about 0.1 cm and about 1 cm, more particularly between about 0.2 cm and about 0.75 cm, more particularly between about 0.25 cm and about 0.5 cm, where the disc has a diameter of between about 0.5 cm and about 12 cm, more particularly between about 1 cm and about 9.5 cm, more particularly about 2 cm and about 7 cm, and more particularly between about 3 cm and about 6 cm. The hydrogel may be visually divided into sections to aid identification of a nexus of infection for the wound. In some embodiments, the hydrogel is marked with lines that divide its surface area into quadrants or other sections.

The hydrogel 102 is generally transparent and is flexible to provide direct contact with the wound surface area. In various embodiments, the hydrogel 102 is composed of a natural polymer, a synthetic polymer, or a combination thereof. Suitable natural polymers include gelatin, collagen, agar-agar, hyaluronic acid, chitosan, heparin, alginate, fibrin, bacterial nanocellulose, and combinations or copolymers thereof. Gelatin, for example, provides an excellent safety profile and malleability and is well-suited for adsorbing the pH-indicating dye 104. Suitable synthetic polymers include polyvinyl alcohol, polyethylene glycol, sodium polyacrylate, polyvinylpyrrolidone, and combinations or copolymers thereof. Different polymer concentrations may be used in various embodiments, e.g., to obtain the desired flexibility for the pH-sensitive wound dressing 100 or to facilitate adsorption of the pH-indicating dye 104 by the hydrogel 102. In various embodiments, the hydrogel 102 is biocompatible, absorbs excess fluid, and demonstrates flexibility based on the selected natural and/or synthetic polymer(s).

The hydrogel 102 optionally incorporates an anti-microbial agent. The hydrogel 102 may also optionally incorporate one or more acidic components to aid in wound healing. In such embodiments where the hydrogel 102 incorporates an anti-microbial agent and/or acidic components to aid in wound healing, these additional components should not interfere with the pH-dependent color change of the pH-indicating dye 104, thereby masking the detection of wound infection (i.e., by producing a false negative or a false positive).

The hydrogel 102 may also incorporate additives that improve adsorption surface area or retention of the pH-indicating dye 104 within the hydrogel 102. In an exemplary embodiment, a dye-retention additive employs polyethylenimine capping to improve adsorption of the pH-indicating dye 104. In another exemplary embodiment, the hydrogel 102 incorporates silica nanoparticles (before or after adsorption of the pH-indicating dye 104) to increase dye retention.

The hydrogel 102 facilitates contact between the pH-indicating dye 104 and the wound. As depicted in FIGS. 1A and 1B, in some embodiments, the hydrogel 102 is covered by a layer of the pH-indicating dye 104 after the hydrogel 102 solidifies, after which the pH-indicating dye 104 is permitted to adsorb into the hydrogel 102 for a pre-determined adsorption period (e.g., 5 minutes, 10 minutes, 20 minutes, 30 minutes, 45 minutes, 1 hour). In other embodiments, the pH-indicating dye 104 is mixed directly into the hydrogel 102 before solidification of the hydrogel solution.

The pH-indicating dye 104 demonstrates a color change over a known pH range. In a non-limiting embodiment, the pH-indicating dye 104 has a high-contrast colorimetric change as a function of pH in the range of pH 4 to 9, which allows for visual detection of pH shifts from normal and non-infected skin (pH of 4 to 6) to infected skin (pH of 7 to 9). Suitable pH-indicating dyes 104 include bromothymol blue, azolitmin, neutral red, methylene blue, and combinations thereof. Bromothymol blue, for example, demonstrates a drastic color change of non-physiological colors (bright yellow to bright blue) from a pH of about 5 to a pH of about 8. More particularly, bromothymol blue absorbs light at around 430 nm to be yellow in color in acidic solutions below pH 6.0 and absorbs light at around 600 nm to 620 nm to be blue in color in alkaline solutions around or above pH 7.5. Although bromothymol blue exhibits a yellow-to-blue color change, it will be appreciated that other non-physiological color changes (e.g., from green to blue) are suitable for detection on a range of human skin tones. In various embodiments, two or more pH-indicating dyes 104 are used to demonstrate a color change at different indicating pH ranges. In embodiments with two pH-indicating dyes 104, the color change of one pH-indicating dye 104 should not mask the color change of the other pH-indicating dye 104.

In various embodiments, the hydrogel covering 106 offers a protective cover and support for the hydrogel 102, as well as visual access to the pH-indicating dye 104. As such, the hydrogel covering 106 allows a color change of the pH-indicating dye 104 to be monitored without removal of the pH-sensitive wound dressing 100 from the wound. In embodiments of the pH-sensitive wound dressing 100 that include the hydrogel covering 106, the hydrogel covering 106 facilitates continued contact of the hydrogel 102 with the wound by securing the pH-sensitive wound dressing 100 on the patient's body.

The hydrogel covering 106 may be transparent, translucent, or opaque. In certain embodiments where the hydrogel covering 106 is translucent or opaque, the hydrogel covering 106 includes a window or other transparent opening that permits visualization of the hydrogel 102; in other embodiments, the translucent or opaque hydrogel covering 106 must be removed from the hydrogel 102 to evaluate the color of the pH-indicating dye 104.

The hydrogel covering 106 may be made from polyurethane or another elastic material. In several non-limiting embodiments, the hydrogel covering 106 is an adhesive transparent film that is incorporated with the hydrogel 102 or that is placed over the hydrogel 102 after it is positioned against the wound. In other non-limiting embodiments, the hydrogel covering 106 incorporates an adhesive backing that is configured to either contact the hydrogel 102 directly or to contact a perimeter region surrounding the hydrogel 102. In yet other embodiments, the hydrogel covering 106 is a dressing that is laid atop or wrapped against the hydrogel 102 and secured in place with an adhesive tape. The hydrogel covering 106 may also be water-proof or water-resistant to protect the hydrogel 102, the pH-indicating dye 104, and/or the wound from moisture.

In another aspect, a method is disclosed for monitoring a wound for infection. The method involves positioning the pH-sensitive wound dressing 100 in direct contact with the wound. The method also includes the steps of maintaining contact between the pH-sensitive wound dressing 100 and the wound for an observation period and evaluating whether the pH-sensitive wound dressing 100 reflects a color change. The step of maintaining contact between the pH-sensitive wound dressing 100 and the wound is optionally performed by securing the pH-sensitive wound dressing 100 against the wound with, for example, the hydrogel covering 106. The observation period begins upon initial contact of the wound and the pH-sensitive wound dressing 100 and may range from a few seconds to several days. In various embodiments, the observation period ranges from one second after the initial contact to 100 hours after the initial contact, more particularly from 5 minutes to 72 hours (3 days), more particularly from 1 hour to 48 hours, more particularly from 5 hours to 24 hours, more particularly about 10 hours. During the observation period, the pH-sensitive wound dressing 100 is left in place over the wound. The pH-sensitive wound dressing 100 is periodically or continuously evaluated during the observation period for a color change. In one embodiment, the pH-sensitive wound dressing 100 is checked at a pre-determined observation interval (e.g., every hour) for a color change. In one embodiment, the pH-sensitive wound dressing 100 is evaluated for the color change through direct visual observation by the patient, a healthcare professional, or another individual. In another embodiment, a colorimeter, a color strip, or other color-detecting equipment is used to quantitatively evaluate the pH-sensitive wound dressing 100 for color change. The step of evaluating whether the pH-sensitive wound dressing reflects a colorimetric change is optionally performed by comparing the color of the pH-indicating dye to a reference color scale (not shown) by visual inspection. The reference color scale depicts the colors that the pH-indicating dye 104 is expected to create at pre-specified levels of pH (e.g., at pH 4 vs. pH 7). The patient, healthcare professional, or other observer may evaluate the pH-sensitive wound dressing 100 by comparison against this reference color scale to aid in detecting a shift in pH levels. In some embodiments, the upper surface of the hydrogel covering 106 is printed with the reference color scale to provide a reference for interpreting the color of the pH-indicating dye 104. In other embodiments, the reference color scale is provided to the patient on a separate card, a cellphone application, or other easily accessible medium.

To ensure the reliability of pH readings over time, the pH-sensitive wound dressing 100 may be replaced with a second pH-sensitive wound dressing 100 after a predetermined contact period (e.g., 1 day, 2 days, 3 days, 4 days, etc.).

### EXAMPLES

The system and method for monitoring a wound for infection with a pH-sensitive wound dressing is further illustrated by the following Examples, which are provided for the purpose of demonstration rather than limitation.

### Example 1

The goal of this round of testing was to evaluate a cost-effective pH-sensitive wound dressing that could be used to identify wound SSIs for all skin color types. More particularly, collagen, agar-agar, and gelatin were tested at different concentrations as hydrogel materials for the pH-sensitive wound dressing.

Hydrogels were prepared by pouring 3 mL of a hydrogel solution (collagen, agar-agar, or gelatin) into a circular silicone mold 3.75 cm in diameter to create discs that were sized to cover a medium-sized simulated wound. The hydrogels were subsequently infused with bromothymol blue (BTB) as the pH-indicating dye.

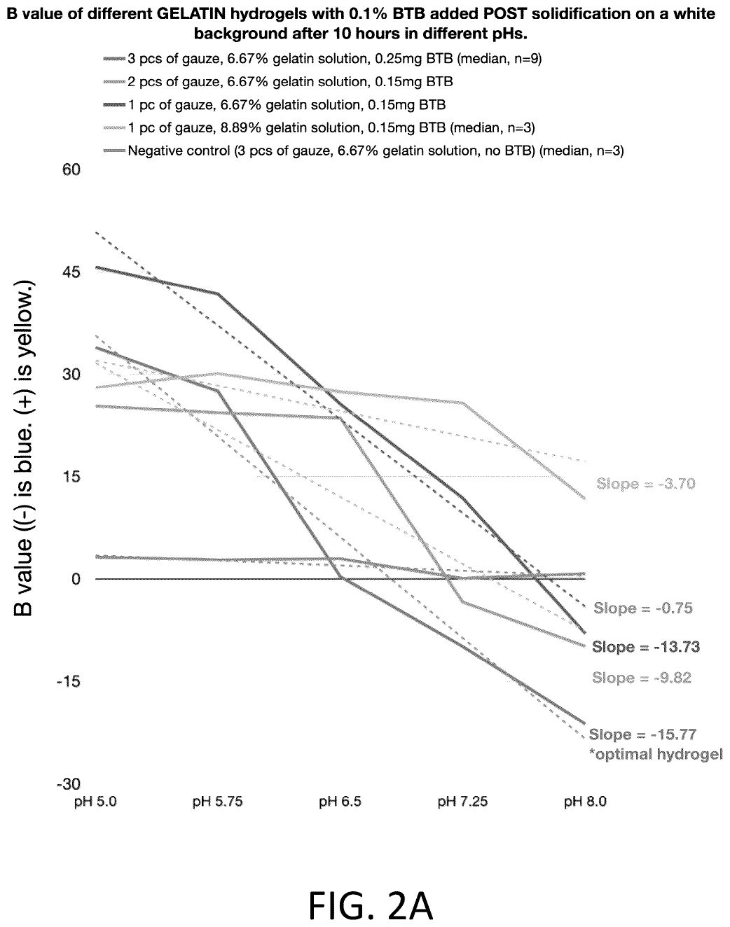

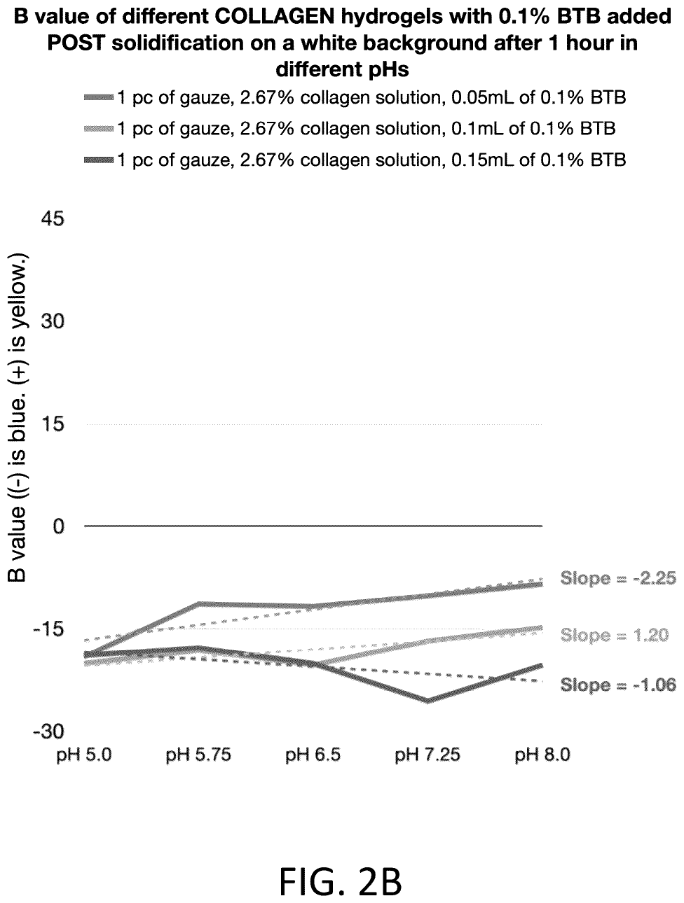

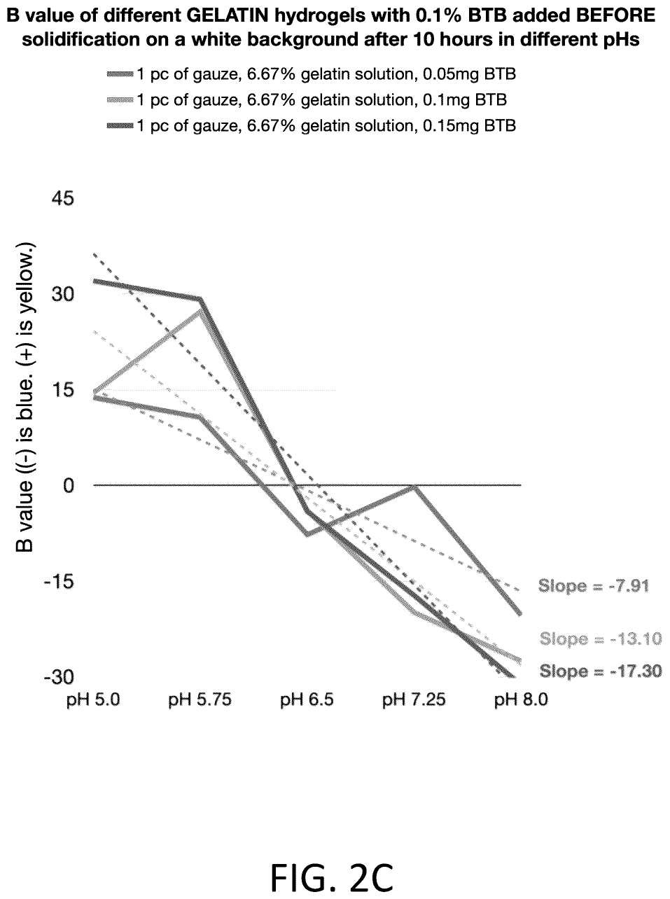

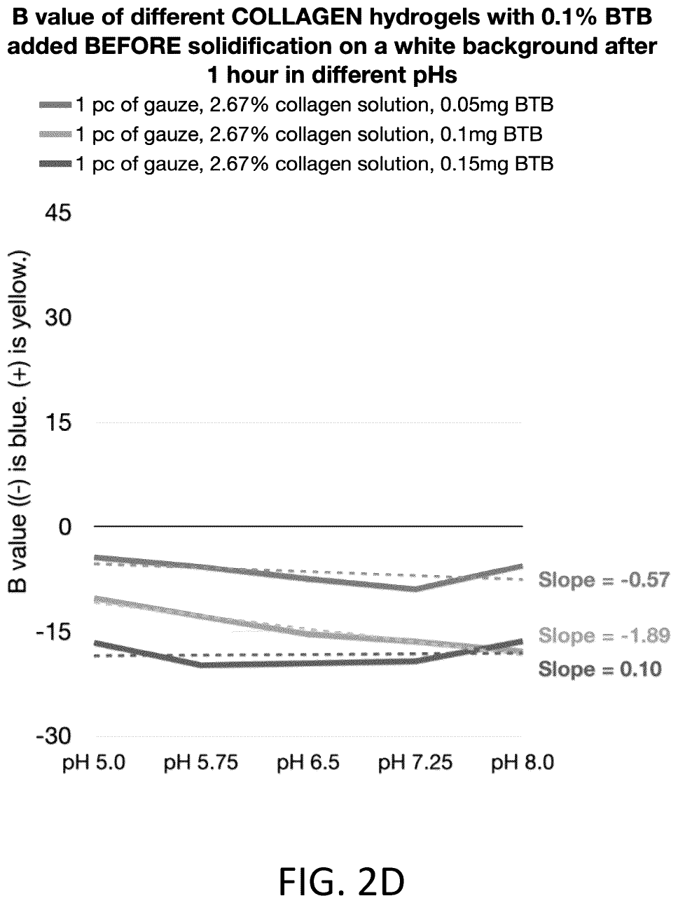

Of the tested hydrogel materials, gelatin infused with BTB demonstrated the most significant range of color and could retain its circular disc shape while still conforming to the wound bed. In contrast, none of the hydrogels made using collagen produced a yellow color unless placed in a very acidic environment. These poor collagen results were not improved by varying the method of incorporating the BTB with the hydrogel, the concentration of collagen, or the concentration of BTB (pH 2.0) (see FIGS. 2B and 2D). The existing pH of the collagen may have interfered with the ability for the BTB to adapt to the pH of a simulated wound (gauze). Another possibility is that the collagen affected the molecular structure of BTB. The hydrogels made using agar-agar were not mechanically robust and lacked the flexibility that the gelatin and collagen hydrogels exhibited, which prevented them from being removed from the mold.

With regard to hydrogel concentration, the hydrogels made using a higher concentration of gelatin (8.89% gelatin solution) did not exhibit as great color differential compared to using a lower concentration of gelatin (6.67% gelatin solution). More particular, the hydrogels made using 8.89% gelatin solution did not produce a blue color change when progressing from pH 5.0 to pH 8.0. In summary, the optimal hydrogel disc from Example 1 was prepared using 3 mL of a 6.67% gelatin solution.

### Example 2

The following tests evaluated the incorporation of a 0.1% solution of bromothymol blue (BTB), which had a molarity of 1.6 mM (based on molar mass of 624.284 g/mol), with a hydrogel to produce a pH-sensitive wound dressing. More particularly, the amount of BTB per hydrogel and various methods of incorporating the BTB with the hydrogel solution (before or after solidification) were evaluated. BTB was selected as the pH-indicating dye due to its striking color change from yellow to green to brilliant blue as the pH increases from 5.5 to 8.0.

In a first set of batches, the hydrogel was incubated at room temperature for 15 minutes to solidify. Once the hydrogel solidified, a layer of BTB was evenly distributed to the surface of the hydrogel, and the hydrogel sat for 30 minutes for the pH-indicating dye to be adsorbed. In other batches, the pH-indicating dye was mixed into the hydrogel before solidification. The amount of BTB per hydrogel was varied (0.05 mg, 0.1 mg, 0.15 mg, 0.25 mg).

When mixing in the BTB before the gelatin had set, the hydrogels still successfully increased in B-value as the pH increased; however, it was difficult to incorporate the BTB evenly, as the BTB became light in color in the gelatin, and the distribution of BTB throughout the hydrogel was uneven (FIG. 2C). The difference between B-values of the BTB-infused hydrogels from the lowest pH (5.0) to the highest pH (8.0) increased as the amount of BTB per disc was increased (FIG. 2A).

### Example 3

After the optimal hydrogel consistency and BTB concentration for the greatest color change were determined as in Examples 1 and 2, the optimal BTB-infused gelatin hydrogel was remade and retested in nine (9) separate repeat experiments to demonstrate reproducibility. The 6.67% gelatin hydrogel allowed excellent contact with a simulated wound because it was malleable and conformed to irregular surfaces. Excellent contact allowed for thorough read out for pH change for the entire wound and possible identification of the nidus of infection.

The following tests evaluated the visibility of the color change for BTB in a gelatin hydrogel against different skin colors. Gauze was used to simulate a wound bed, and different amounts of gauze were varied to simulate different moisture levels in the wound bed. More particularly, pieces of circular gauze (1.5 inches in diameter) were soaked in different concentrations of vinegar (for acidic pHs) and baking soda (for basic pHs) and water to create solutions with pHs of 5.0, 5.75, 6.5, 7.25, and 8.0. More gauze (and therefore more moisture in the wound bed) allowed more visually detectable color differential (from yellow to blue) because all the B-values in pH 8.0 using more gauze were more blue (B-value was more negative).

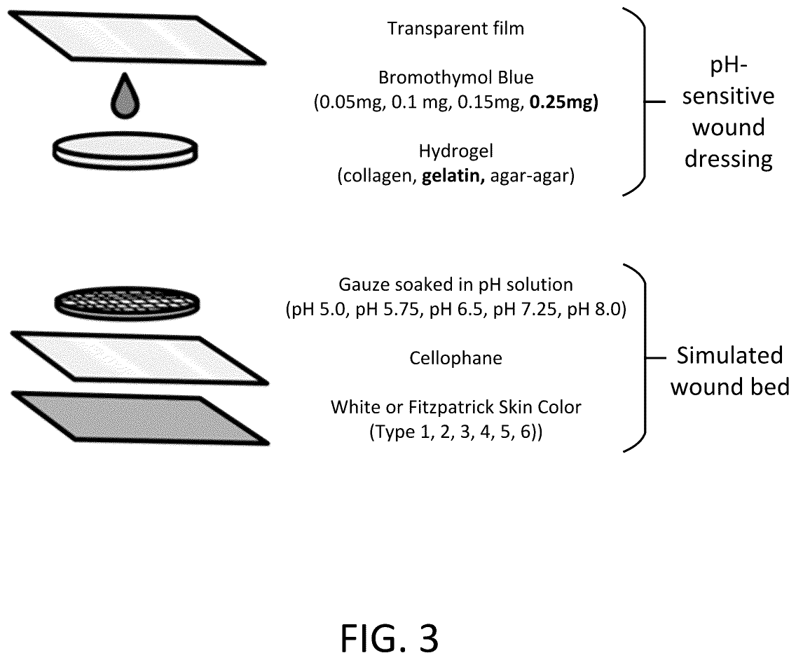

As shown in FIG. 3, the gauze was placed on clear cellophane covering paper shaded with Fitzpatrick skin colors to simulate skin wounds. The six (6) skin color types in the Fitzpatrick Scale that were used hard varying levels of darkness (Type 1=fairest; Type 6=darkest). The hydrogel was placed over the simulated wound site (soaked gauze) and then a transparent hydrogel covering was placed over the hydrogel to allow easy visualization and measurement of color change without removal of the dressing. The transparent hydrogel covering also prevented the wound and hydrogel desiccation. The slope of the color change (B-value; positive=yellow, negative=blue) color was recorded in the center of the hydrogel after 5 mins, 10 hours, 24 hours, and 100 hours using a colorimeter. The B-values were compared to find the hydrogel with the greatest change in color. After the optimal color change was determined, a nonparametric test (the Kruskal-Wallis test) was used to find the lowest pH that exhibited a significant statistical change in B-value compared to pH 5.0. The colorfastness was also graphed to determine the longevity of the readout.

In these tests, pH test strips with a detection range from pH of 4.5 to 9.0 were initially planned as the positive control. However, pH test strip results tended to be somewhat variable with a very ephemeral readout period and less accuracy given their relatively low resolution. Given the limitations of pH test strips and the need for accurate measurement of pH in a relatively narrow range of pH 5.0-8.0, a pH meter was used instead as a positive control. The pH 5.0, 5.75, 6.5, 7.25, and 8.0 solutions were tested using a pH meter immediately before submerging the gauze in the solutions. The negative control for each round of testing was a hydrogel without any BTB on a piece of gauze dipped in a pH solution. The negative control hydrogels showed almost no change in B-value (slope −0.80, median, n=3) as the pH increased.

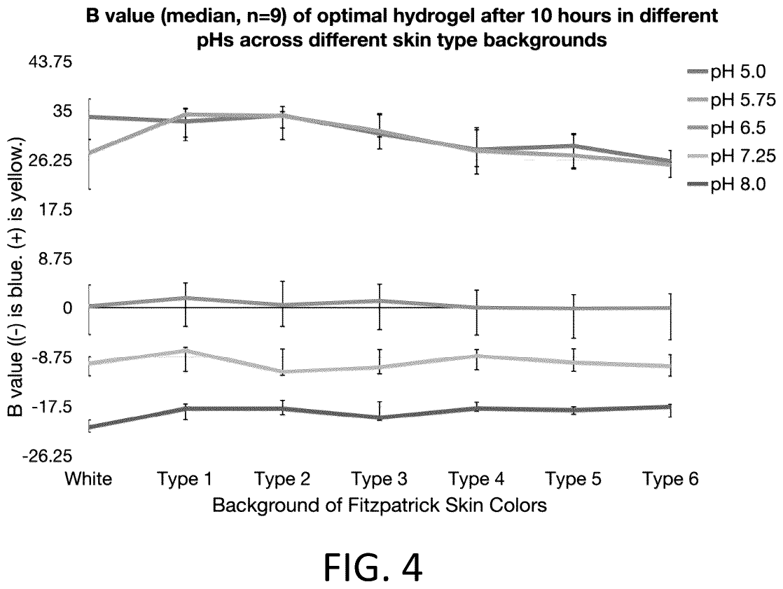

FIG. 4 depicts the B-values (median, n=9) of a series of hydrogels with pH-indicating dye (BTB) after 10 hours, where each of the hydrogels was made with 5 grams of gelatin in 75 mL of distilled water and 5 drops of 0.1% BTB added after solidification. The slope of the trend line of median B-values of the optimal hydrogel on a white background from pH 5.0 to pH 8.0 of these 9 trials was −15.77. The slope of the trend line of median B-values of the hydrogel on a background of Type 1 Fitzpatrick skin color from pH 5.0 to pH 8.0 of these 9 trials was −14.39. The slope of the trend line of median B-values of the hydrogel on a background of Type 2 Fitzpatrick skin color from pH 5.0 to pH 8.0 of these 9 trials was −14.96. The slope of the trend line of median B-values of the hydrogel on a background of Type 3 Fitzpatrick skin color from pH 5.0 to pH 8.0 of these 9 trials was −14.27. The slope of the trend line of median B-values of the hydrogel on a background of Type 4 Fitzpatrick skin color from pH 5.0 to pH 8.0 of these 9 trials was −12.84. The slope of the trend line of median B-values of the hydrogel on a background of Type 5 Fitzpatrick skin color from pH 5.0 to pH 8.0 of these 9 trials was −13.06. The slope of the trend line of median B-values of the hydrogel on a background of Type 6 Fitzpatrick skin color from pH 5.0 to pH 8.0 of these 9 trials was −12.30 (see Table 1).

TABLE 1
B-value (median, n = 9) of hydrogel
after 10 hours across different pHs

| | Slope of trend line from |
|---|---|
| Fitzpatrick skin type | pH 5.0 to pH 8 |
| White | −15.77 |
| Type 1 | −14.39 |
| Type 2 | −14.96 |
| Type 3 | −14.27 |
| Type 4 | −12.84 |
| Type 5 | −13.06 |
| Type 6 | −12.30 |

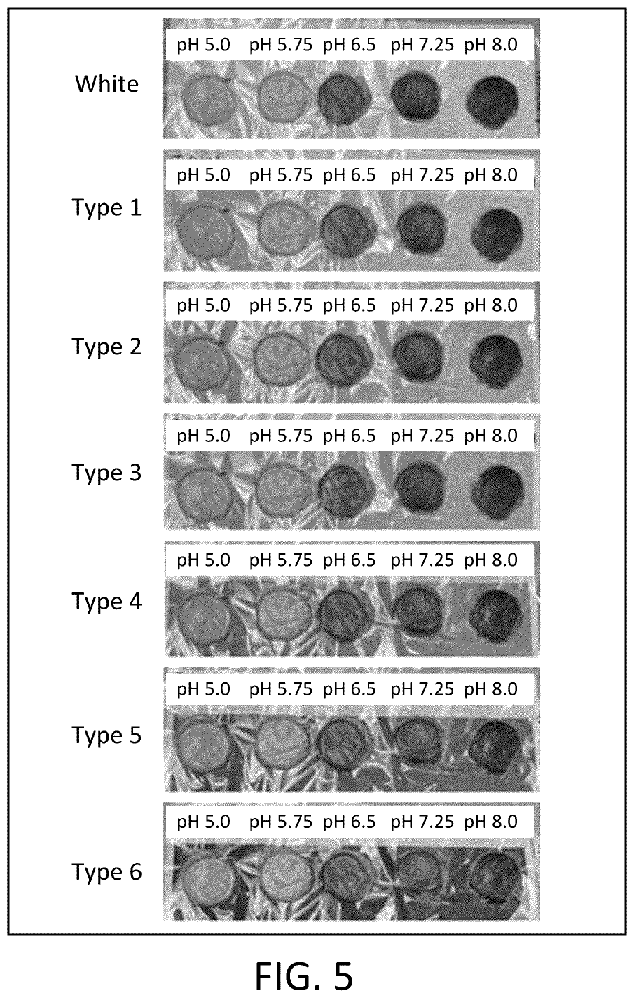

As the Fitzpatrick skin color scale background became darker, the slope decreased only minimally, indicating that there was only minimally less contrast between the B-value of pH 5.0 and pH 8.0. The greatest median B-value of pH 8.0 was −17.58 on Type 4 Fitzpatrick skin color, and the least median B-value of pH 8.0 was −21.43 on the white background. The B-value of pH 5.0 decreased as the background became darker, meaning the hydrogels appeared less yellow. The greatest median B-value at pH 5.0 was 33.60 on white background, and the least median B-value of pH 5.0 was 27.08 on Type 6 Fitzpatrick skin color. Thus, the BTB-infused hydrogels allow detection of a visually significant color change from pH 5.0 to pH 8.0 in the white and all 6 Fitzpatrick skin color backgrounds. Throughout all backgrounds, the B-value of the hydrogels in pH 5.0 and 5.75 had relatively similar positive B-values and were very yellow. The B-value in pH 6.5 was around 0, and the hydrogel was either slightly yellow or slightly blue. The B-value of pH 7.25 and 8.0 were both blue (FIG. 5).

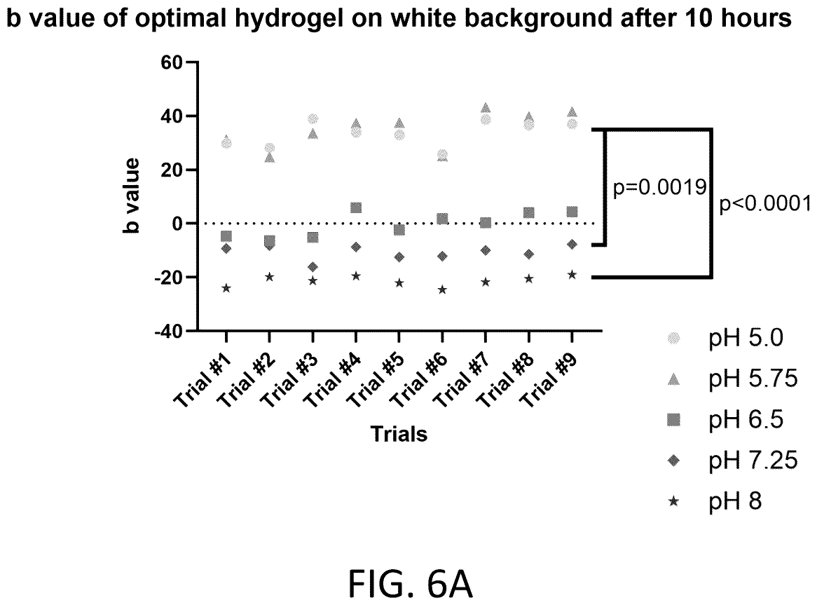

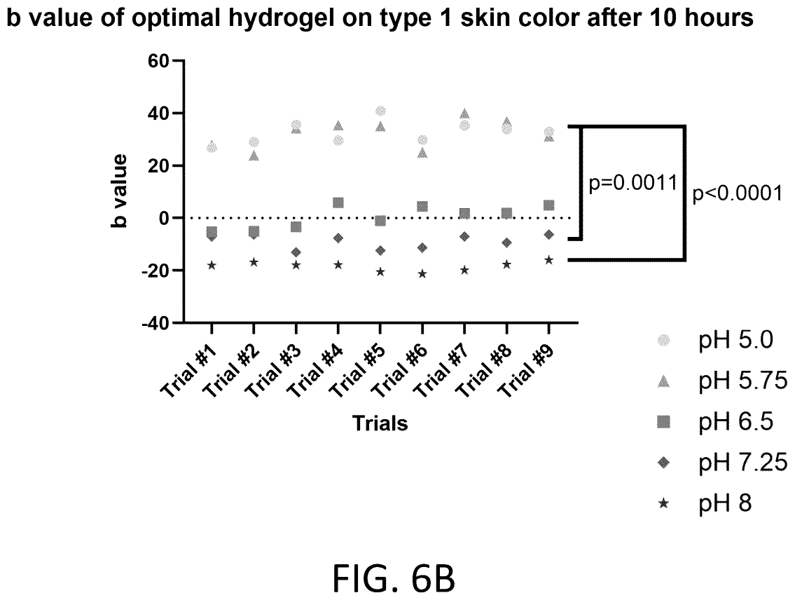

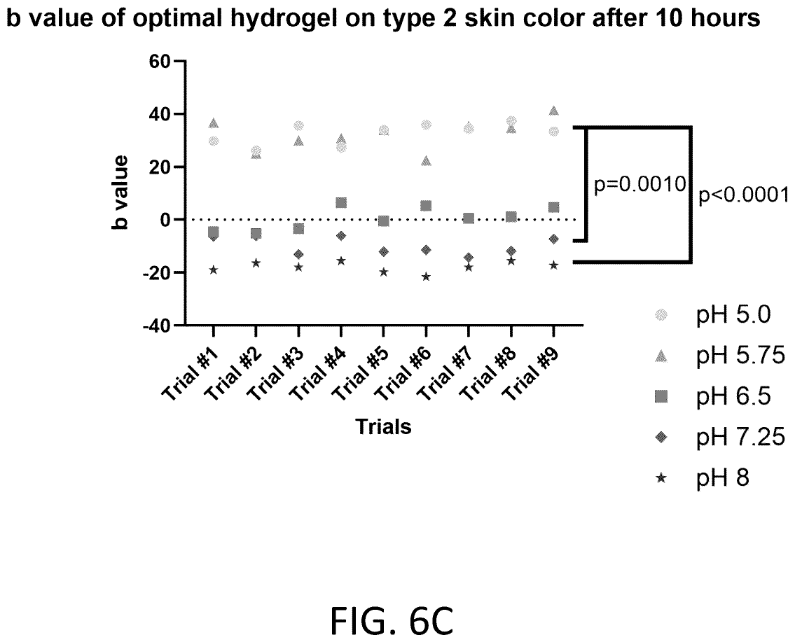

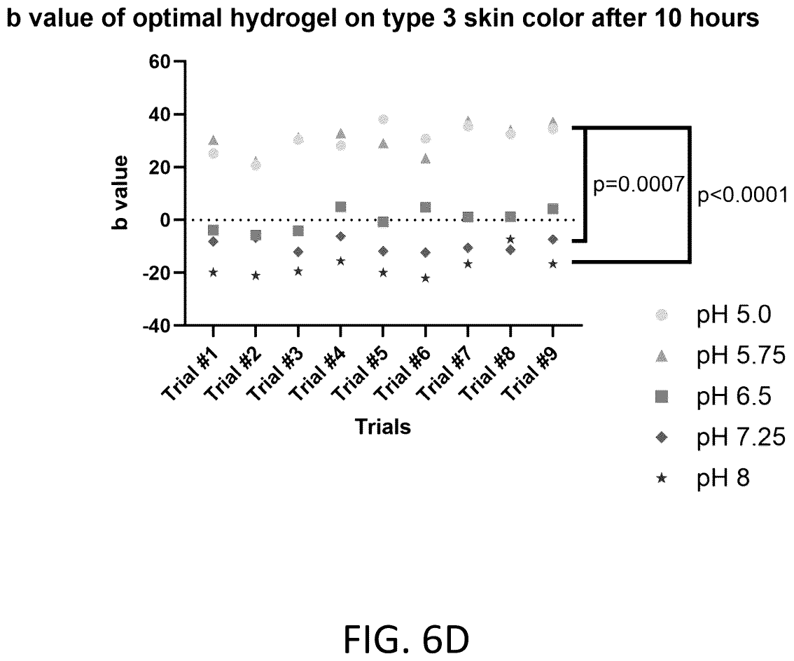

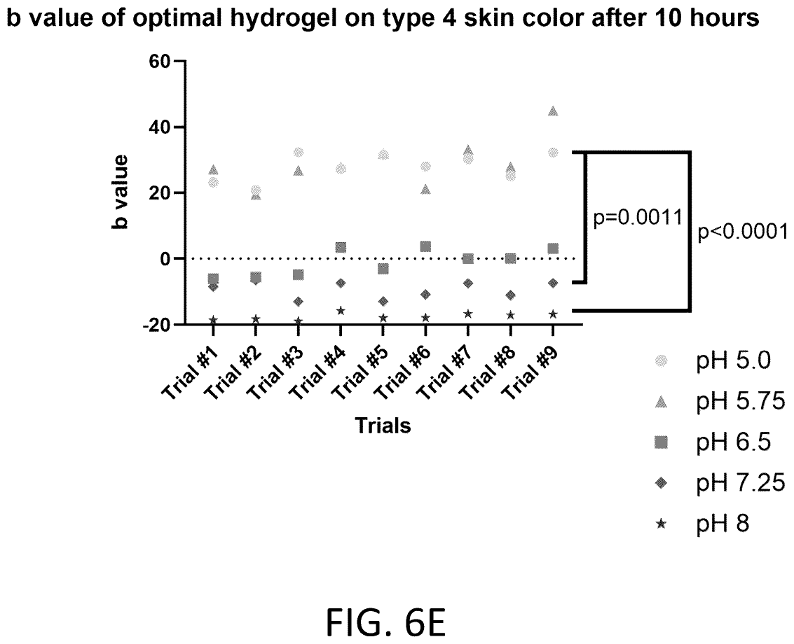

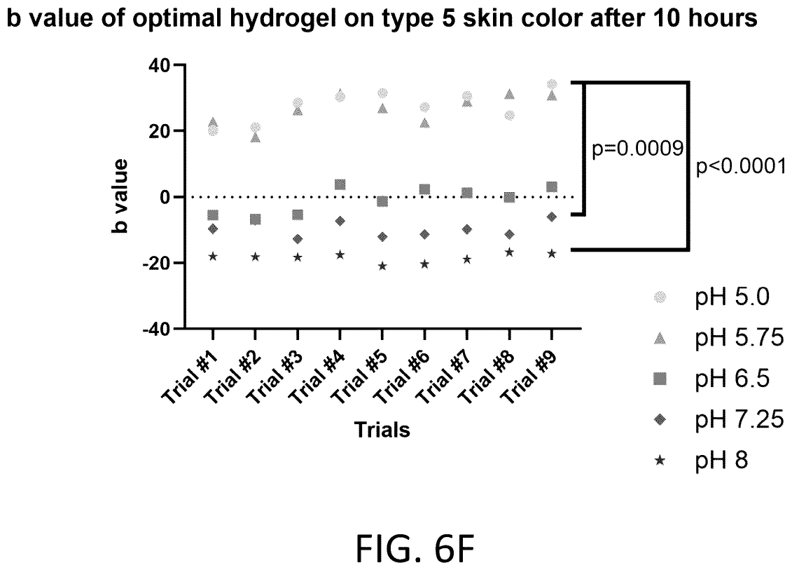

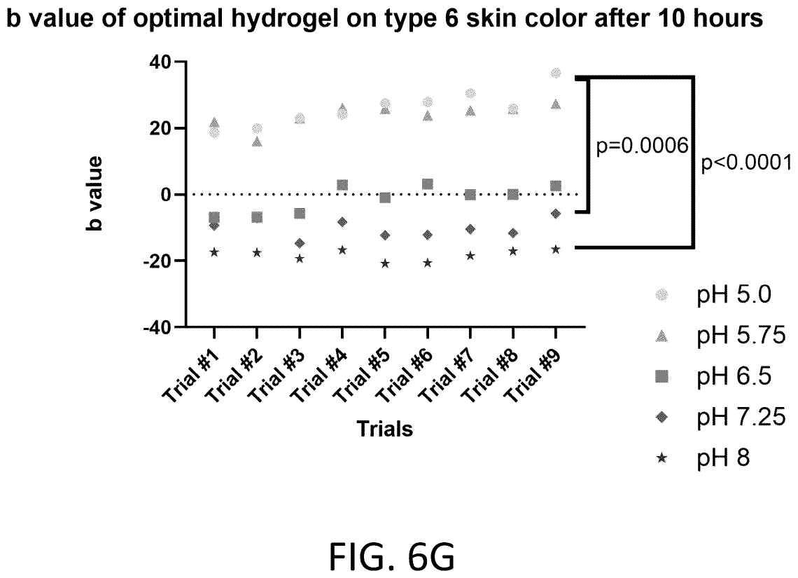

The Kruskal-Wallis test was used to find the lowest pH for which there was a significant statistical change in B-value compared to the B-value in pH 5.0. It was found that there was a significant statistical change of the optimal hydrogel in pH 5.0 and hydrogel in pH 7.25 and pH 8.0 on white and all 6 skin colors of the Fitzpatrick scale. P values less than 0.05 were considered significant (see FIG. 6).

FIG. 6A depicts the gelatin hydrogel with BTB on a white background after 10 hours. On a white background, the difference in B-value of pH 5.0 and pH 7.25 was statistically significant with a p value of 0.0019. The difference in B-value of pH 5.0 and pH 8.0 was statistically significant with a p value <0.0001. The difference in B-value of pH 5.0 vs pH 5.75 and pH 5.0 vs pH 6.5 was not statistically significant.

FIG. 6B depicts the B-value of the gelatin hydrogel with BTB on Type 1 Fitzpatrick skin color after 10 hours. On a background of Type 1 Fitzpatrick skin color, the difference in B-value of pH 5.0 and pH 7.25 was statistically significant with a p value of 0.0011. The difference in B-value of pH 5.0 and pH 8.0 was statistically significant with a p value of <0.0001. The difference in B-value of pH 5.0 vs pH 5.75 and pH 5.0 vs pH 6.5 was not statistically significant.

FIG. 6C depicts B-value of the gelatin hydrogel with BTB on Type 2 Fitzpatrick skin color after 10 hours. On a background of Type 2 Fitzpatrick skin color, the difference in B-value of pH 5.0 and pH 7.25 was statistically significant with a p value of 0.0010. The difference in B-value of pH 5.0 and pH 8.0 was statistically significant with a p value of <0.0001. The difference in B-value of pH 5.0 vs pH 5.75 and pH 5.0 vs pH 6.5 was not statistically significant.

FIG. 6D depicts B-value of the gelatin hydrogel with BTB on Type 3 Fitzpatrick skin color after 10 hours. On a background of Type 3 Fitzpatrick skin color, the difference in B-value of pH 5.0 and pH 7.25 was statistically significant with a p value of 0.0007. The difference in B-value of pH 5.0 and pH 8.0 was statistically significant with a p value of <0.0001. The difference in B-value of pH 5.0 vs pH 5.75 and pH 5.0 vs pH 6.5 was not statistically significant.

FIG. 6E depicts B-value of the gelatin hydrogel with BTB on Type 4 Fitzpatrick skin color after 10 hours. On a background of Type 4 Fitzpatrick skin color, the difference in B-value of pH 5.0 and pH 7.25 was statistically significant with a p value of 0.0011. The difference in B-value of pH 5.0 and pH 8.0 was statistically significant with a p value of <0.0001. The difference in B-value of pH 5.0 vs pH 5.75 and pH 5.0 vs pH 6.5 was not statistically significant.

FIG. 6F depicts B-value of the gelatin hydrogel with BTB on Type 5 Fitzpatrick skin color after 10 hours. On a background of Type 5 Fitzpatrick skin color, the difference in B-value of pH 5.0 and pH 7.25 was statistically significant with a p value of 0.0009. The difference in B-value of pH 5.0 and pH 8.0 was statistically significant with a p value of <0.0001. The difference in B-value of pH 5.0 vs pH 5.75 and pH 5.0 vs pH 6.5 was not statistically significant.

FIG. 6G depicts B-value of the gelatin hydrogel with BTB on Type 6 Fitzpatrick skin color after 10 hours. On a background of Type 6 Fitzpatrick skin color, the difference in B-value of pH 5.0 and pH 7.25 was statistically significant with a p value of 0.0006. The difference in B-value of pH 5.0 and pH 8.0 was statistically significant with a p value of <0.0001. The difference in B-value of pH 5.0 vs pH 5.75 and pH 5.0 vs pH 6.5 was not statistically significant.

The Kruskal-Wallis nonparametric test demonstrated that there was a significant statistical change in B-value in addition to an easily detectible visual change in color from yellow at pH 5.0 compared to blue at pH 7.25 and pH 8.0 throughout all backgrounds, which correlates to the pH of infected wounds (pH 7 to pH 9).

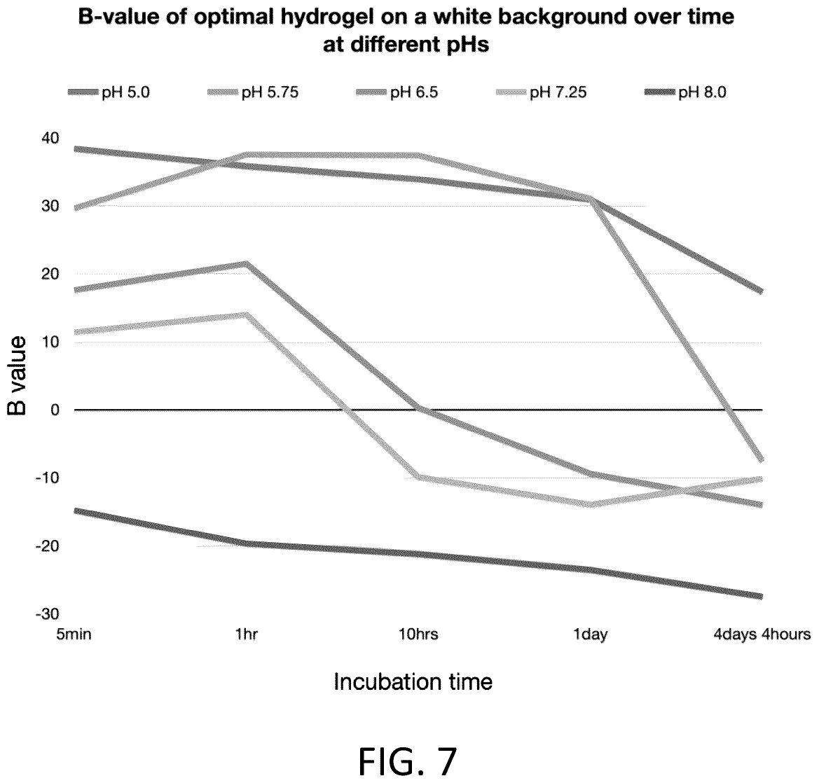

The optimal hydrogel maintained a color differential between the different pHs that could be visually discriminated up to the longest incubation time (100 hours) (see spread of lines in FIG. 7). At 10 hours, the median B-values (n=9) at pH 5.0, 5.75, 6.5, 7.25, and 8.0 were 33.89 (very yellow), 37.44 (very yellow), 0.31 (greenish), −9.93 (blue), and −21.26 (very blue), respectively. At 10 hours, the color readout was most distinguishable between the pHs and likely the most potentially clinically significant given the pH range for an infected surgical site ranges about pH 7-9. For the mid-range pHs of 5.75, 6.5, and 7.25, the B-values tended to decline with incubation time (hydrogels became more blue). After 5 minutes incubation and after 1 hour incubation, the B-values at pHs 6.5 and 7.25 were positive (yellow), but after 10 hours incubation, the B-values at pHs 6.5 and 7.25 dropped closer to zero (became less yellow and more blue), such that it became difficult to visually distinguish the color differential between pH 5.75, 6.5, and 7.25 at 100 h (FIG. 7). Importantly, though, throughout all incubation times (5 minutes, 1 hour, 10 hours, 1 day, 4 days), the B-value for pH 5.0 remained quite positive (hydrogels maintained a yellow color). Additionally, throughout all incubation times (5 minutes, 1 hour, 10 hours, 24 hours, 100 hours), the B-value for pH 8.0 remained quite negative (hydrogels maintained a dark blue color). Over time, all B-values of all hydrogels at all pHs slowly declined, becoming more blue. Given hydrogel dressings were determined to last about 3 days before needing to be changed, the color differential between pHs 5.0 and 8.0 should last the duration of most hydrogel dressings.

In conclusion, the optimal hydrogel consisted of 3 mL of a 0.67% gelatin solution and 0.25 mg BTB added after solidification and showcased the greatest range of B-value from pH 5.0 to pH 8.0 and had a color change that was easily visually detectable and statistically significant (in pH 5.0 compared to pH 7.25 and 8.0) on all 6 Fitzpatrick skin color types. The BTB-infused gelatin hydrogels demonstrated a colorimetric change from pH 5.0 to 8.0 throughout all levels of simulated wound moisture, and it was determined that, of the time frames observed, 10 hours of incubation produced the optimal color readout. The gelatin formed a gentle adhesion to the wound site and would be less likely to damage tissue when removed compared to gauze dressings that dries and adheres to the wound, causing possible damage to the healing wound when removed. This hydrogel was also very cost-efficient, and one hydrogel-based pH-sensitive wound dressing was calculated to be $0.40.

### Example 4

Additional tests were performed to evaluate the pH sensitivity of the optimized hydrogel from Examples 1-3 (3 mL 6.67% gelatin, circular with 3.75 inches in diameter, 0.25 mg BTB added after solidification). These tests addressed how mild of an infection the pH-sensitive wound dressing was able to detect and the smallest difference in pH required to display a visually detectable and statistically significant change in color. These tests evaluated how early an infection could be detected using the hydrogel (i.e., whether a color change could be detected at the point of initial alkalinization for the wound bed and, if not, the extent of alkalinization of the wound bed required to change the color of the hydrogel).

To set up these tests, three pieces of gauze were cut in half. The first three halves of gauze were stacked on top of each other and dipped in a solution of pH 5.0. The other three halves of gauze were stacked on top of each other and dipped in another pH solution (pH 5.0, pH 5.75, pH 6.5, pH 7.25, pH 8.0). The positive control was three pieces of gauze with both halves dipped into a solution of pH 5.0.

The optimized hydrogel from Examples 1-3 was placed over each gauze, and a colorimeter was used to measure the B-value of the left and rights halves for each hydrogel after 10 hours of incubation and on Type 1 Fitzpatrick skin color and Type 6 Fitzpatrick skin color. The Kruskal-Wallis test with Dunn's multiple comparisons was used to determine the lowest pH that exhibited statistically significant change in B-value from the left side (pH 5.0) to the right side (pH 5.0, 5.75, 6.5, 7.25, or 8.0) of the hydrogel. These tests were repeated in nine separate repeat trials.

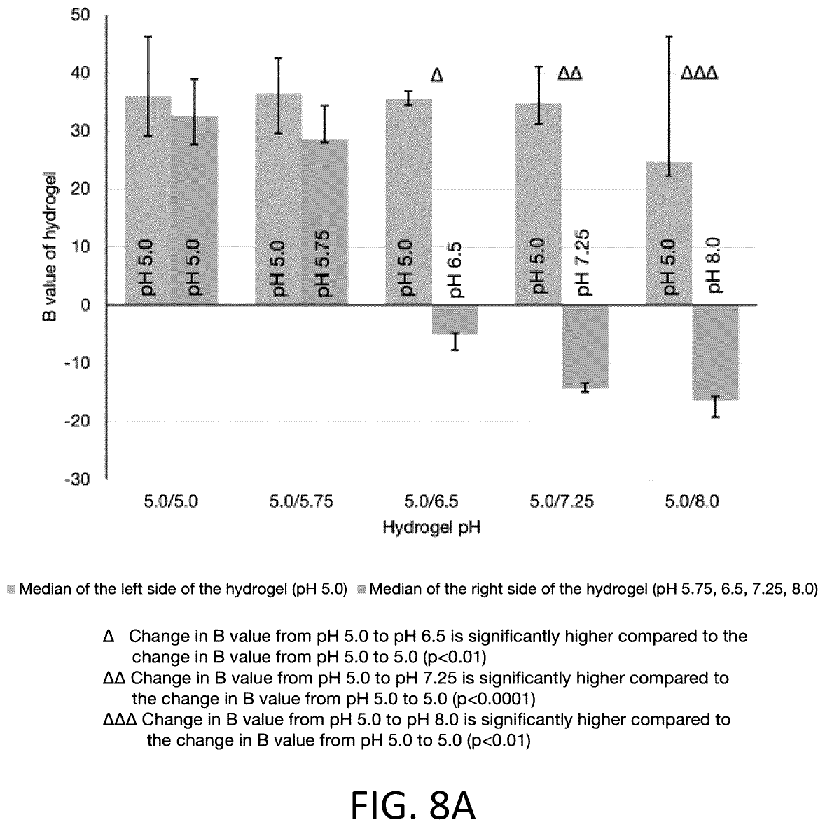

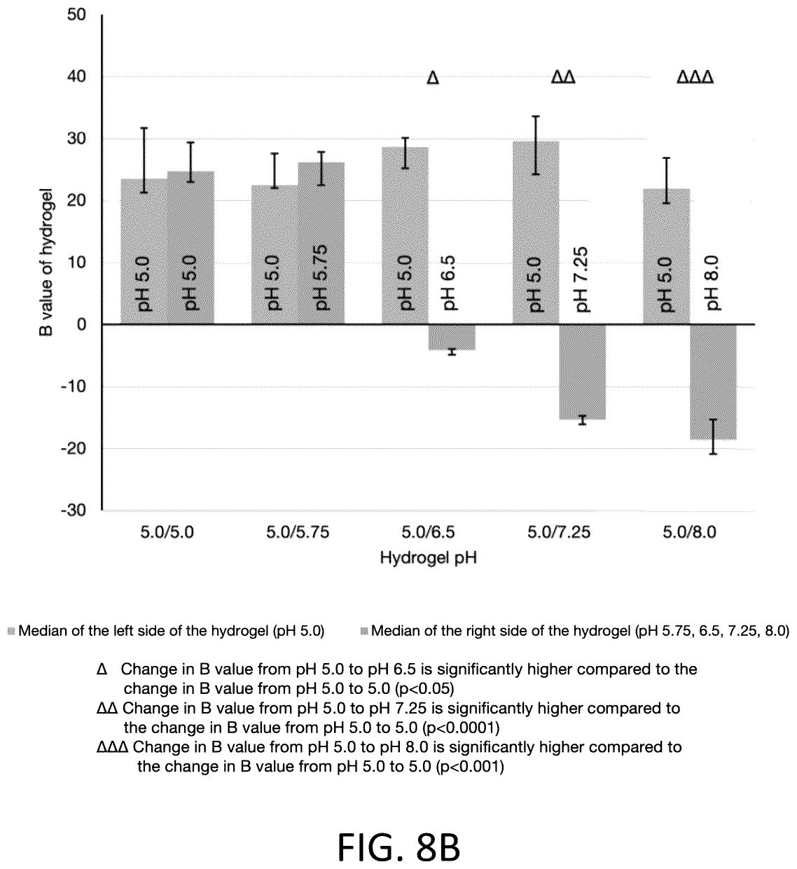

On a background of Type 1 Fitzpatrick skin color (fairest), there was a statistically significant difference in between the median change in B-value from the left side of the hydrogel in pH 5.0 to the right side of the hydrogel in pH 6.5 (p=0.0091), pH 7.25 (p<0.0001), and pH 8.0 (p=0.0015) when compared to the difference in B-value of the left and right sides of the control hydrogel in pH 5.0 (see FIG. 8A). As shown in FIG. 8B, on a background of Type 6 Fitzpatrick skin color (darkest), there were also statistically significant differences between the median change in B-value from the left side of the hydrogel on gauze soaked in pH 5.0 to the right side of the hydrogel on gauze soaked in pH 6.5 (p=0.0352), pH 7.25 (p<0.0001), and pH 8.0 (p=0.0003) compared to the median change in B-value from the control hydrogel soaked in pH 5.0. These results demonstrate that a pH-sensitive wound dressing with this BTB-hydrogel hydrogel could demonstrate visually distinguishable and statistically different colors—from yellow in (pH 5.0) to blue (in pH 7.25 and 8.0)—when there are differences in pH within the same wound bed on Type 1 (fairest) and 6 (darkest) Fitzpatrick skin colors.

### Example 5

Additional localization and spatial sensitivity tests were performed to determine the smallest area needed for the optimized hydrogel of Examples 1-3 to exhibit a visually detectable and statistically significant difference in color due to change in pH. These tests evaluated how small a nidus of infection the pH-sensitive wound dressing could detect and whether it could localize where in the wound bed the infection was starting, thereby allowing for more precise culturing, debridement, or treatment of the wound.

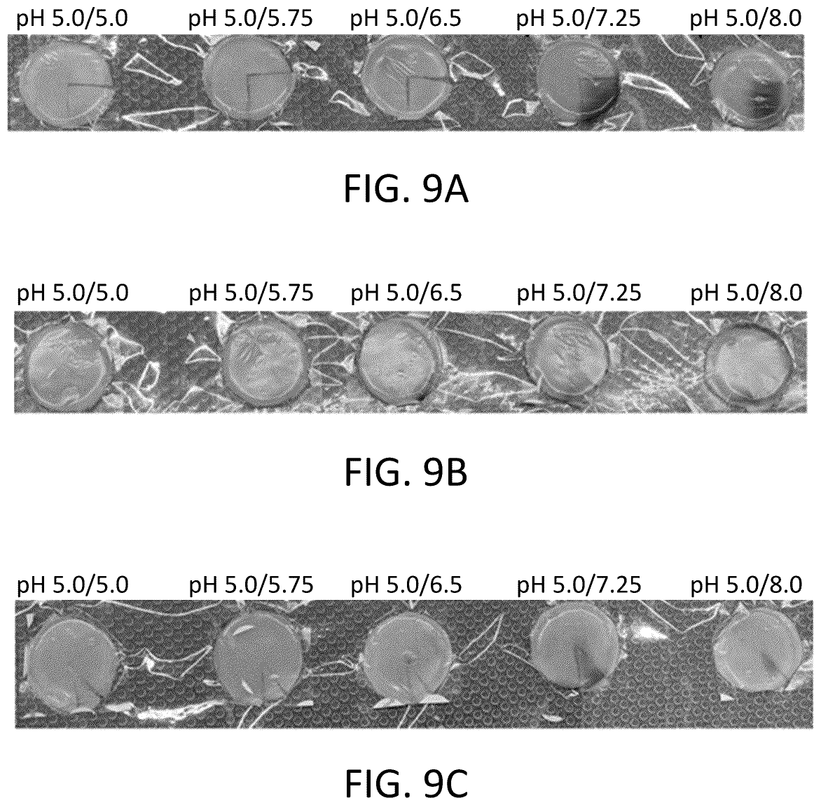

For these tests, three pieces of gauze were cut into two portions each. For the first round of testing, the first portion made up ¾ of the area of the original gauze, and the other portion made up ¼ of the original gauze area (see FIG. 9A). For the second and third rounds of testing, the three pieces of gauze were cut into portions that made up ⅞ and ⅛ of the original gauze area (see FIG. 9B) and portions that made up 15/16 and 1/16 of the original gauze area (FIG. 9C), respectively. In all rounds of testing, the corresponding portions of gauze were stacked on each other and the larger portions (i.e., ¾, ⅞, and 15/16 of the gauze area) were dipped in pH 5.0. The smaller portions (i.e., ¼, ⅛, and 1/16 of the gauze area) were dipped in a pH solution of pH 5.0, pH 5.75, pH 6.5, pH 7.25, or pH 8.0. A colorimeter measured the B-value of the area with pH 5.0 and area with the other pH after 10 hours.

It was found that there was a visually detectable difference in color between the larger part and the smaller part across all sizes. As shown in FIG. 9A, a visually detectable difference in color occurred from yellow in the ¾ piece of gauze to blue in the ¼ piece of gauze dipped in pH 7.25 and 8.0. FIG. 9B depicts a visually detectable difference in color from yellow in the ⅞ piece of gauze to blue in the ⅛ piece of gauze dipped in pH 7.25 and 8.0. FIG. 9C depicts a visually detectable difference in color from yellow in the 15/16 piece of gauze to blue in the 1/16 piece of gauze dipped in pH 7.25 and 8.0. Thus, a pH-sensitive wound dressing with this hydrogel exhibits a yellow-to-blue color change that would allow patients to identify and distinguish the location of an infection as small as 0.69 cm2 in size.

### Example 6

Time sensitivity tests were performed to determine whether the optimized hydrogel of Examples 1-3 would change colors in response to a patient's dynamic wound condition. To accomplish this objective, the tests evaluated the longest time one hydrogel could be placed on a gauze of pH 5.0 before being placed on another gauze of a different pH and still exhibit a visually detectable and statistically significant color change. In other words, these tests investigated whether the pH-sensitive wound dressing would remain dynamic and able to change colors and detect later onset of infection after sitting on a physiologic wound bed for a prolonged time. The purpose of this experiment was to discern if hydrogel's color changing properties could remain active for a long period of time. This would be beneficial if, for example, a patient developed a delayed-onset infection after the initial placement of the hydrogel on the wound bed.

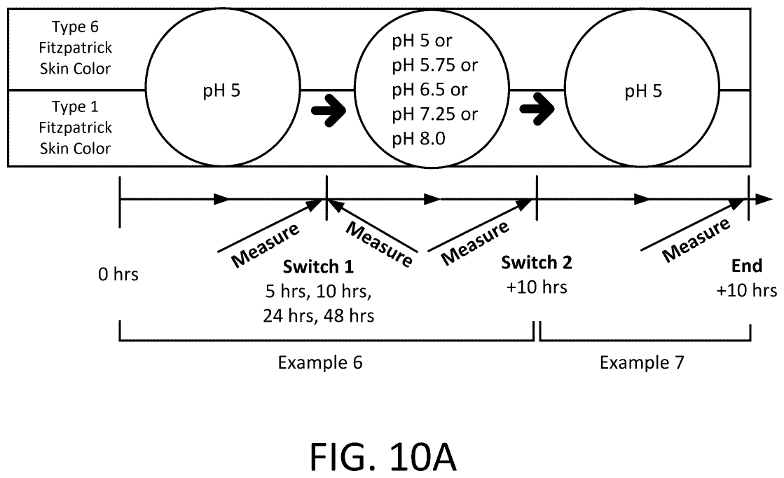

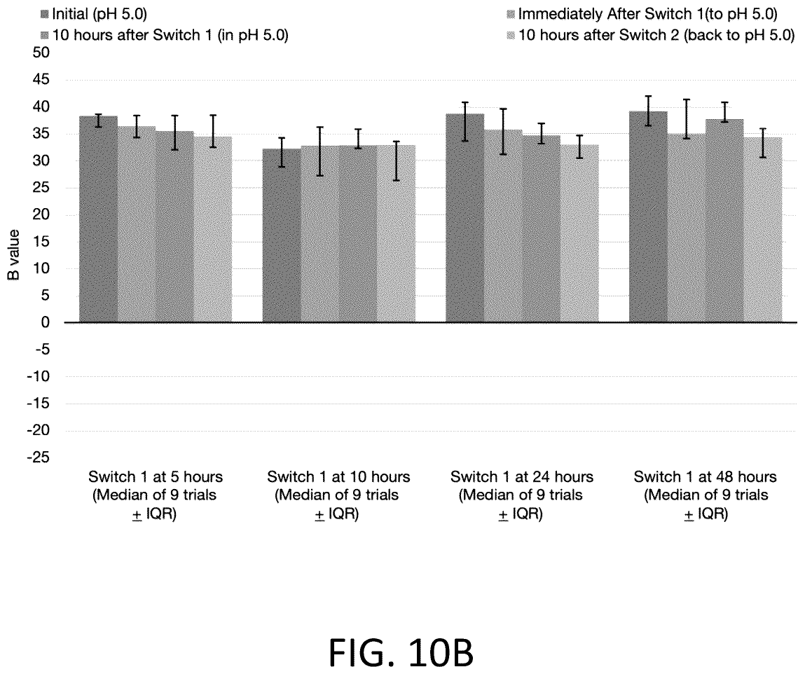

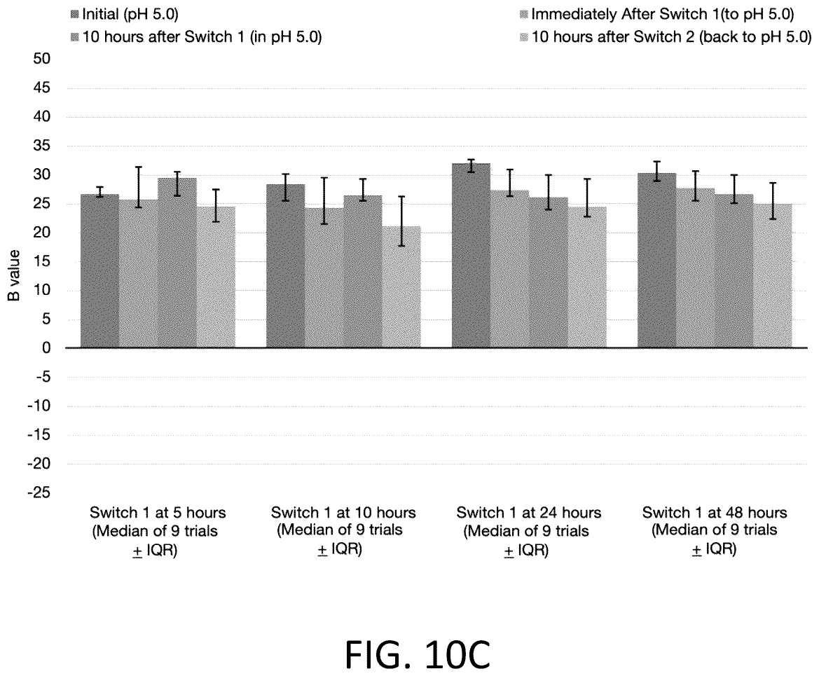

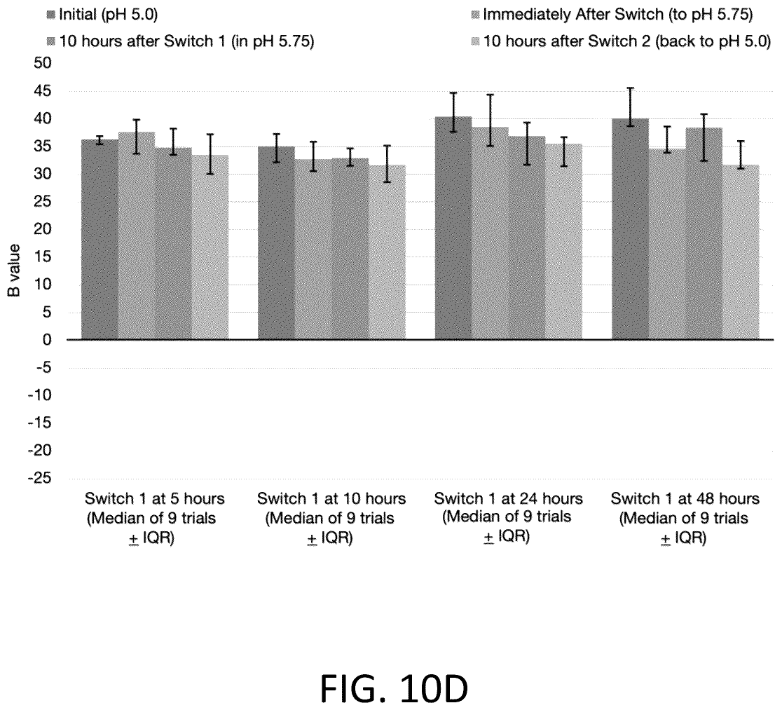

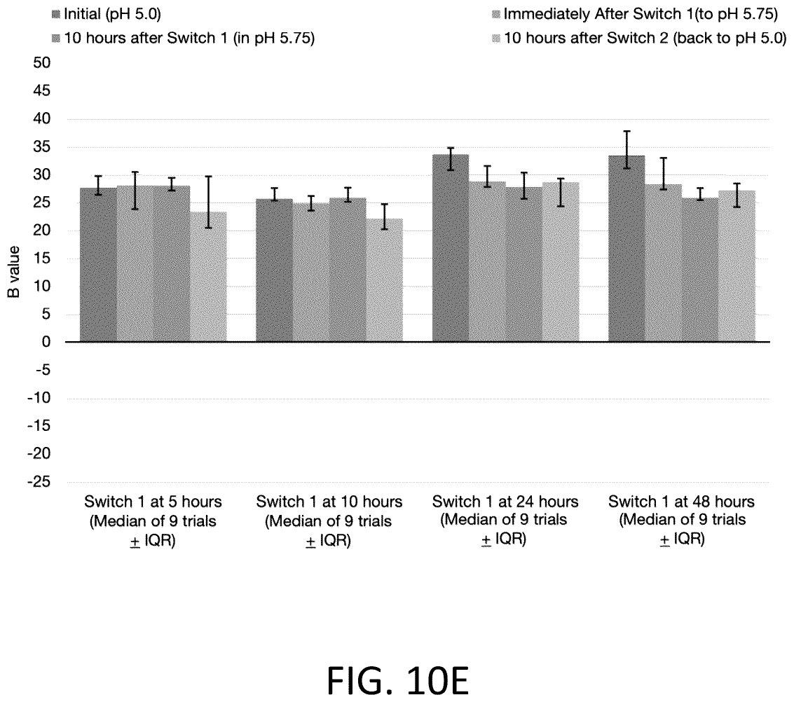

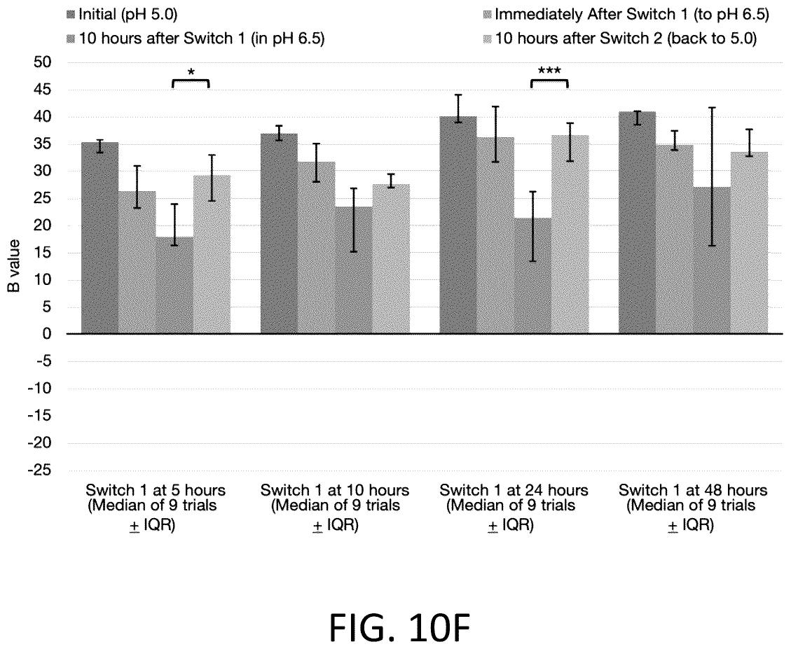

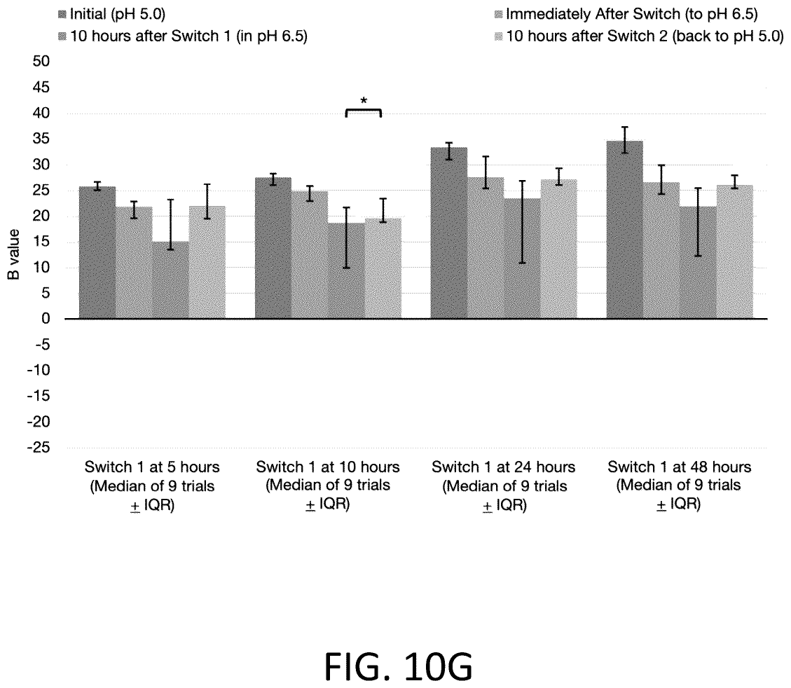

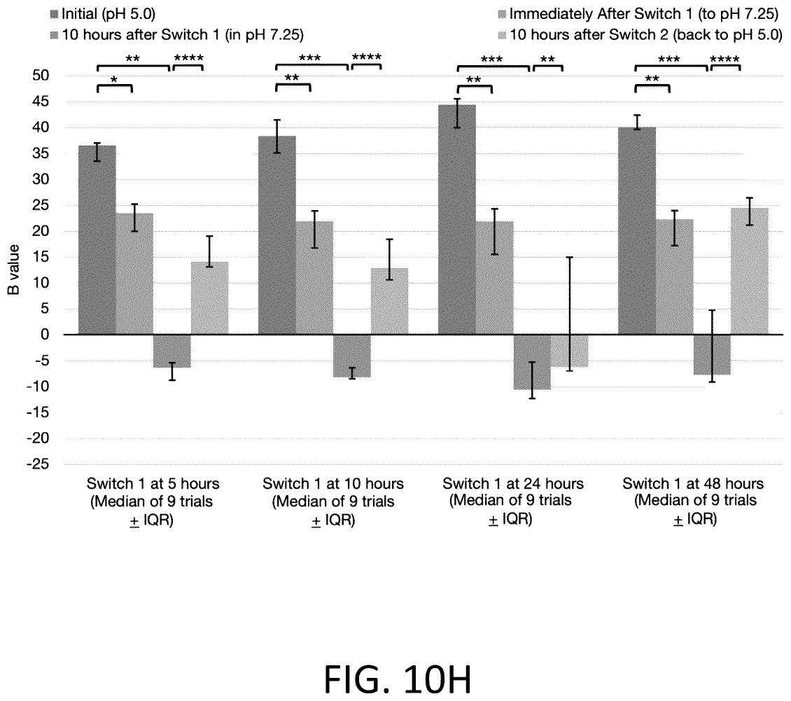

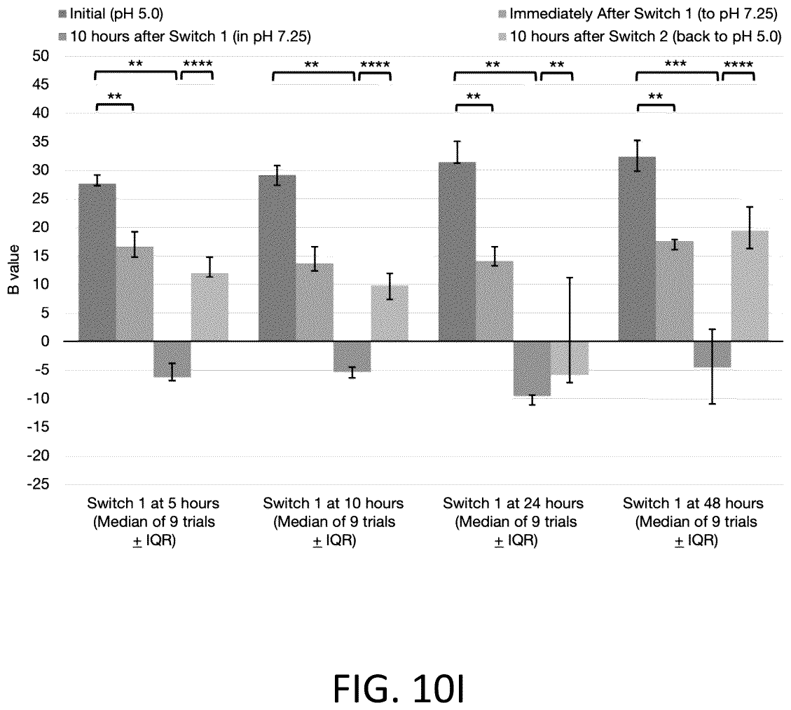

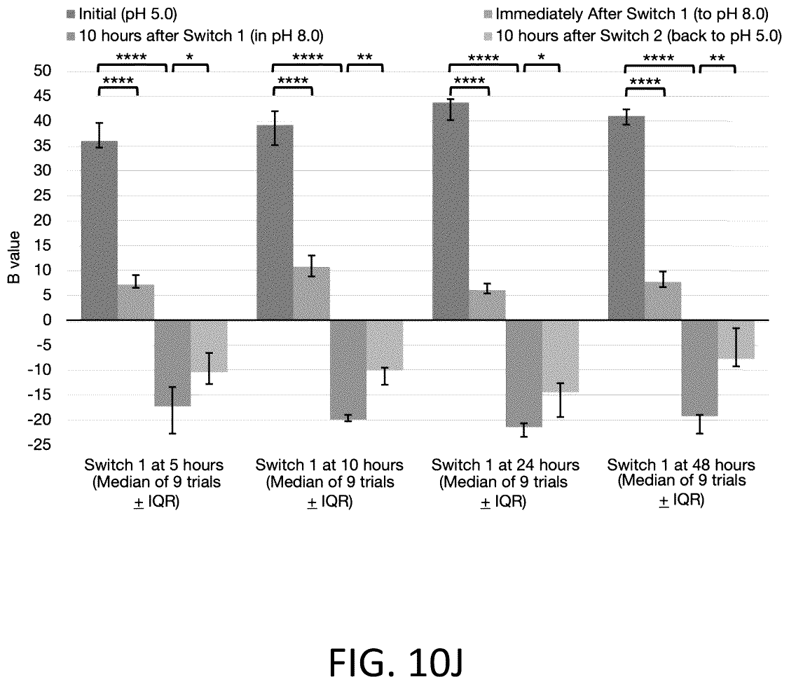

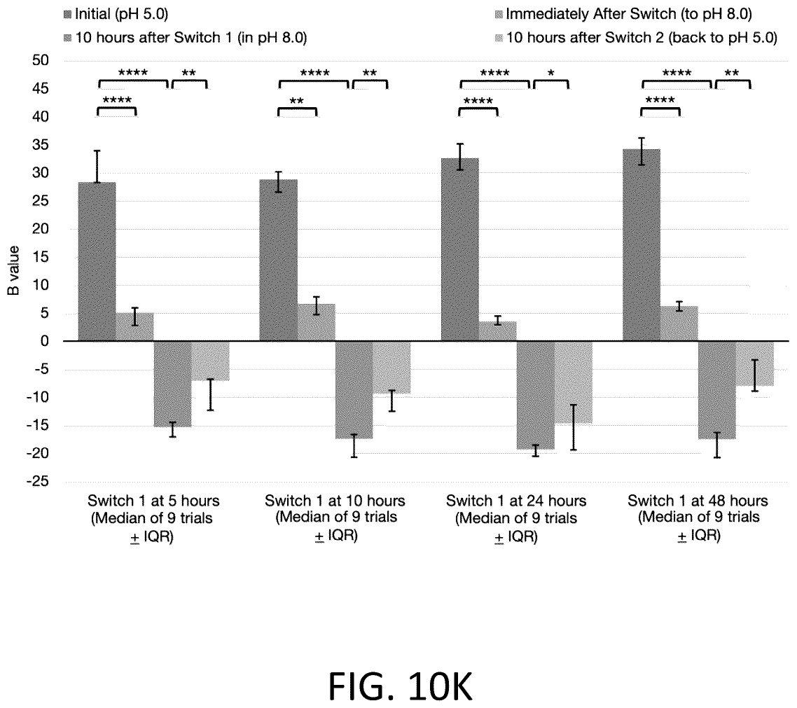

To simulate a non-infected wound bed, hydrogels were placed on three pieces of gauze soaked in a solution of pH 5.0. A piece of transparent film dressing was placed on top of the hydrogel. The hydrogels were left on this pH 5.0 gauze for different amounts of time (5 hours, 10 hours, 24 hours, 48 hours) before transfer to another three pieces of gauze soaked in a solution with a designated pH (pH 5.0, pH 5.75, pH 6.5, pH 7.25, pH 8.0) (“Switch 1”). This set-up simulated an infection developing in the wound bed at 5 hours, 10 hours, 24 hours, or 48 hours after initial placement on the wound bed. The color of the hydrogels was measured with a colorimeter immediately after the transfer and again after 10 hours of incubation (see FIG. 10A).

The Kruskal-Wallis test with Dunn's multiple comparisons was used to determine the lowest pH that exhibited a difference between the B-value of the hydrogel in pH 5.0 and the B-value of the same hydrogel after being switched to a pH (5.0, 5.75, 6.5, 7.25, or 8.0) that was statistically significantly greater than the difference between the B-value of the hydrogel in pH 5.0 and the B-value of the same hydrogel after being switched to a pH of 5.0. As shown in FIG. 10B, on a background of Type 1 Fitzpatrick skin color (fairest), there were no differences seen in B-value with the changes of pH (from 5.0 to 5.0 back to 5.0) at different time points. Similarly, as shown in FIG. 10C, on a background of Type 6 Fitzpatrick skin color (darkest), there were no differences seen in B-value with the changes of pH (from 5.0 to 5.0 back to 5.0) at different time points. The difference between the B-value of the hydrogel in pH 5.0 and the B-value of the same hydrogel after being switched to a pH of 7.25 or 8.0 (see FIGS. 10H, 10I, 10J, and 10K) was statistically significantly greater than the difference between the B-value of the hydrogel in pH 5.0 and the B-value of the same hydrogel after being switched to a pH of 5.0 (see FIGS. 10B and 10C). Similar differences were seen in all time points of pH switches, which extended as far as 48 hours. These statistically significant differences were immediately noted after the change in pH and were replicated with a background of both Type 1 (fairest) and Type 6 (darkest) Fitzpatrick skin colors.

Thus, this Example 6 determined that if an infection was to develop up to 48 hours after initial placement of the hydrogel wound dressing on the wound bed (start of incubation), the BTB-hydrogel hydrogel could still immediately exhibit a visually detectable and statistically significant color change from yellow to blue on Type 1 and 6 Fitzpatrick skin colors.

### Example 7

Reversibility tests explored whether the pH-sensitive wound dressing could continue to detect more than one direction of pH change and go through multiple color changes as infection waxes and wanes. More particularly, these tests were performed to determine if the optimized hydrogel of Examples 1-3 could exhibit a statistically significant color change from an acidic pH (simulating physiologic conditions) to an alkaline pH (simulating infection) back to an acidic pH (simulating a healed wound bed). Reversibility of the pH detection system would be beneficial, for example, if a patient with an infected wound bed received treatment, and the infected alkaline wound bed returned to a normal healthy acidic pH. By monitoring for a reverse in color change, a patient could evaluate their recovery progress in addition to detecting the initial infection.

The same hydrogels from Example 6 were used for this Example 7. After the hydrogels from Example 6 were incubated for 10 hours at the designated pH (pH 5.0, pH 5.75, pH 6.5, pH 7.25, or pH 8.0), the hydrogels were transferred to three new pieces of gauze soaked in a solution of pH 5.0 (“Switch 2’). As shown in FIG. 10A, the color of the hydrogel was measured using the colorimeter after the initial 10 hours of incubation and again after the 10 additional hours of incubation (for a total of 25 hours, 30 hours, 44 hours, 68 hours).

The Kruskal-Wallis test with Dunn's multiple comparisons was used to determine the lowest pH that exhibited a difference between the B-value of the hydrogel in a pH (5.0, 5.75, 6.5, 7.25, or 8.0) and the B-value of the same hydrogel after being switched back to pH 5.0 that was statistically significantly greater than the difference between the B-value of the hydrogel in pH 5.0 and the B-value of the same hydrogel after being switched back to a pH of 5.0. The differences in B-value from initial to immediately after Switch 1, the differences in B-value from initial to 10 hours after Switch 1, and the differences in B-value from 10 hours after Switch 1 to 10 hours after Switch 2 were used as reference values for the Dunn's multiple comparisons test and Kruskal Wallis test.

As shown in FIGS. 10D and 10E, there were no differences seen in B-value with the changes of pH (from 5.0 to 5.75 back to 5.0) at different time points on, respectively, Type 1 Fitzpatrick skin color (fairest) and Type 6 Fitzpatrick skin color (darkest). FIG. 10F depicts that on a background of Type 1 Fitzpatrick skin color (fairest), the difference between the B-value of the hydrogel 10 hours after Switch 1 in pH 6.5 and the B-value of the hydrogel 10 hours after Switch 2 switched back to pH 5.0 was statistically significantly greater compared to the difference between the B-value of the hydrogel 10 hours after Switch 1 in pH 5.0 and the B-value of the hydrogel 10 hours after Switch 2 back to pH 5.0. These differences were seen in the hydrogels where Switch 1 occurred at 5 hours (p=0.0316) and 24 hours (p=0.0009). As shown in FIG. 10G, on a background of Type 6 Fitzpatrick skin color (darkest), the difference between the B-value of the hydrogel 10 hours after Switch 1 in pH 6.5 and the B-value of the hydrogel 10 hours after Switch 2 switched back to pH 5.0 was statistically significantly greater compared to the difference between the B-value of the hydrogel 10 hours after Switch 1 in pH 5.0 and the B-value of the hydrogel 10 hours after Switch 2 back to pH 5.0. These differences were seen in the hydrogels where Switch 1 occurred at 10 hours (p=0.0308).

The difference between the B-value of the hydrogel in pH 7.25 and 8.0 and the B-value of the same hydrogel after being switched back to pH 5.0 (see FIGS. 10H, 10I, 10J, and 10K) was statistically significantly greater than the difference between the B-value of the hydrogel in pH 5.0 and the B-value of the same hydrogel after being switched back to a pH of 5.0 (see FIGS. 10B and 10C). Similar reversibility characteristics were seen in all time points of pH switches. With reference to FIG. 10H, on a background of Type 1 Fitzpatrick skin color (fairest), the difference between the B-value of the hydrogel in pH 5.0 and the B-value of the same hydrogel after being switched to a pH of 7.25 was statistically significantly greater than the difference between the B-value of the hydrogel in pH 5.0 and the B-value of the same hydrogel after being switched to a pH of 5.0. Similar differences were seen in all time points of pH switches, which extended as far as 48 hours. These differences were seen immediately after Switch 1 (5 hours: p=0.0109, 10 hours: p=0.0014, 24 hours: p=0.0034, 48 hours: p=0.0032) and 10 hours after Switch 1 (5 hours: p=0.0016, 10 hours: p=0.0002, 24 hours: p=0.0001, 48 hours: p=0.0009). Additionally, the difference between the B-value of the hydrogel in pH 7.25 and the B-value of the same hydrogel after being switched back to pH 5.0 was statistically significantly greater than the difference between the B-value of the hydrogel in pH 5.0 and the B-value of the same hydrogel after being switched back to a pH of 5.0. Similar reversibility characteristics were seen in all time points of pH switches (5 hours: p<0.0001, 10 hours: p<0.0001, 24 hours: p=0.0028, 48 hours: p<0.0001).

Turning to FIG. 10I, on a background of Type 6 Fitzpatrick skin color (darkest), the difference between the B-value of the hydrogel in pH 5.0 and the B-value of the same hydrogel after being switched to a pH of 7.25 was statistically significantly greater than the difference between the B-value of the hydro-gel in pH 5.0 and the B-value of the same hydrogel after being switched to a pH of 5.0. These differences were seen immediately after Switch 1 (5 hours: p=0.0081, 24 hours: p=0.0027, 48 hours: p=0.0019) and 10 hours after Switch 1 (5 hours: p=0.0020, 10 hours: p=0.0032, 24 hours: p=0.0017, 48 hours: p=0.0003). Additionally, the difference between the B-value of the hydrogel in pH 7.25 and the B-value of the same hydrogel after being switched back to pH 5.0 was statistically significantly greater than the difference between the B-value of the hydrogel in pH 5.0 and the B-value of the same hydrogel after being switched back to a pH of 5.0. Similar reversibility characteristics were seen in all time points of pH switches (5 hours: p<0.0001, 10 hours: p<0.0001, 24 hours: p=0.0032, 48 hours: p<0.0001).

Turning to FIG. 10J, on a background of Type 1 Fitzpatrick skin color (fairest), the difference between the B-value of the hydrogel in pH 5.0 and the B-value of the same hydrogel after being switched to a pH of 8.0 was statistically significantly greater than the difference between the B-value of the hydrogel in pH 5.0 and the B-value of the same hydrogel after being switched to a pH of 5.0. Similar differences were seen in all time points of pH switches, which extended as far as 48 hours. These differences were seen immediately after Switch 1 (5 hours: p<0.0001, 10 hours: p<0.0001, 24 hours: p<0.0001, 48 hours: p<0.0001) and 10 hours after Switch 1 (5 hours: p<0.0001, 10 hours: p<0.0001, 24 hours: p<0.0001, 48 hours: p<0.0001). Additionally, the difference between the B-value of the hydrogel in pH 7.25 and the B-value of the same hydrogel after being switched back to pH 5.0 was statistically significantly greater than the difference between the B-value of the hydrogel in pH 5.0 and the B-value of the same hydrogel after being switched back to a pH of 5.0. Similar reversibility characteristics were seen in all time points of pH switches (5 hours: p=0.0390, 10 hours: p=0.0097, 24 hours: p=0.0138, 48 hours: p=0.0056).

Lastly, as depicted in FIG. 10K, on a background of Type 6 Fitzpatrick skin color (darkest), the difference between the B-value of the hydrogel in pH 5.0 and the B-value of the same hydrogel after being switched to a pH of 8.0 was statistically significantly greater than the difference between the B-value of the hydrogel in pH 5.0 and the B-value of the same hydrogel after being switched to a pH of 5.0. Similar differences were seen in all time points of pH switches, which extended as far as 48 hours. These differences were seen immediately after Switch 1 (5 hours: p<0.0001, 10 hours: p=0.0010, 24 hours: p<0.0001, 48 hours: p<0.0001) and 10 hours after Switch 1 (5 hours: p<0.0001, 10 hours: p<0.0001, 24 hours: p<0.0001, 48 hours: p<0.0001). Additionally, the difference between the B-value of the hydrogel in pH 7.25 and the B-value of the same hydrogel after being switched back to pH 5.0 was statistically significantly greater than the difference between the B-value of the hydrogel in pH 5.0 and the B-value of the same hydrogel after being switched back to a pH of 5.0. Similar reversibility characteristics were seen in all time points of pH switches (5 hours: p=0.0091, 10 hours: p=0.0017, 24 hours: p=0.0316, 48 hours: p=0.0063).

For Example 7 (as well as Example 6), B-value decreased over time even when the hydrogel was kept in a pH 5.0 simulated wound bed for the entirety of the experiment (see FIGS. 10B and 10C). This decrease may have occurred because the BTB dye in the hydrogel transferred to the gauze, resulting in a decrease in BTB concentration in the hydrogel as the gauzes were switched.

In summary, Example 7 exhibited that the color of the BTB-gelatin hydrogel could be reversed from blue to yellow from an infected alkaline pH to a normal acidic pH after showing a statistically significant change in color from yellow to blue (healthy to infected). The tested hydrogel exhibited a statistically significant difference and visually detectable reversal in color in a pH 7.25 or 8.0 simulated wound bed switched back to a pH 5.0 simulated wound bed. Its color changing properties remained active for at least 68 hours after initial placement of the hydrogel on Type 1 and 6 Fitzpatrick skin colors.

### Example 8

Animal model tests were performed to determine if the optimized hydrogel of Examples 1-3 would exhibit a statistically significant color change when placed in different pHs (pH 5.0, pH 5.75, pH 6.5, pH 7.25, or pH 8.0) on ex vivo porcine skin samples. Example 8 explored whether the hydrogel and BTB remained stable in complex animal tissues with buffers and proteases. Porcine skin samples were selected due to the similarities between pig skin and human skin and their comparable wound healing characteristics.

The upper layers of skin were excised to the level of the dermis, and a 3-cm diameter split thickness wound bed was created in each porcine skin samples using a hemostat and blade. A syringe was used to fill the hole with 1 mL of pH solution (pH 5.0, pH 5.75, pH 6.5, pH 7.25, or pH 8.0). A hydrogel as placed on top of the solution. At 5 minutes, 1 hour, 10 hours, and 100 hours after the hydrogel was placed on the wound bed, the color of the hydrogel was measured by the colorimeter.

After three trials, it was determined that the pH solution diffused out of the wound bed so that the wound bed became dry. To ameliorate this problem, a piece of gauze soaked in a designated pH solution (pH 5.0, pH 5.75, pH 6.5, pH 7.25, or pH 8.0) was placed in the wound bed to maintain the pH of the wound bed. In all three trials using this model, the hydrogel displayed a change in color from yellow in pH 5.0 to blue in pH 8.0, but this range in color did not last, as all the hydrogels became blue at 100 hours. It is hypothesized that this color change occurred because the porcine skin samples were left at room temperature for 100 hours, which could have caused spoilage and the proliferation of bacteria. As pork spoils, a shift in color from bright red to dark green also occurs, which would decrease B-value and cause a more blue appearance. Certain ammonia-releasing bacteria, such as Pseudomonas, have also been identified in pork, and ammonia raises pH. The presence of ammonia-releasing bacteria in the skin samples could explain why the hydrogels all became blue over time.

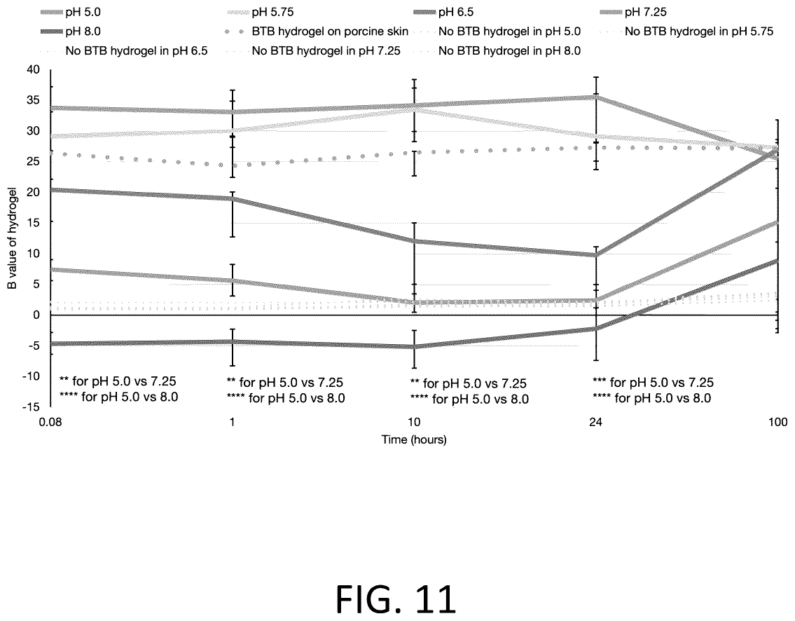

For the next five trials, the porcine wound bed-dressing complexes were vacuum sealed to remove oxygen from the environment and thereby slow bacteria growth that could confound the pH readout. The Kruskal-Wallis test with Dunn's multiple comparisons was used to determine the lowest pH that exhibits a statistically significant difference from the median B-value of the hydrogel in that pH compared to a the B-value of the hydrogel in pH 5.0. These samples demonstrated a statistically significant difference from the median B-value of the hydrogel in pH 5.0 to the median B-value of the hydrogel in pH 7.25 and 8.0. This difference was statistically significant at the 5 minutes, 1 hour, and 10 hour, and 24 hour time points after placement of the hydrogel, as shown in FIG. 11. This animal model demonstrates that on a surface similar to human skin, the hydrogel exhibited a visually distinguishable color change from yellow to blue.

In summary, the BTB-hydrogel exhibited a statistically significant and visually detectable color change in a pH 7.25 or 8.0 simulated wound bed compared to in a pH 5.0 simulated wound bed on ex vivo porcine skin samples.

The description of the invention is intended to be read in connection with the accompanying drawings, which are to be considered part of the entire written description of this invention. In the description, relative terms such as “front,” “rear,” “lower,” “upper,” “horizontal,” “vertical,” “above,” “below,” “up,” “down,” “top” and “bottom” as well as derivatives thereof (e.g., “horizontally,” “downwardly,” “upwardly” etc.) should be construed to refer to the orientation as then described or as shown in the drawings under discussion. These relative terms are for convenience of description and do not require that the machine be constructed or the process to be operated in a particular orientation. Terms, such as “connected,” “connecting,” “attached,” “attaching,” “join” and “joining” are used interchangeably and refer to one structure or surface being secured to another structure or surface or integrally fabricated in one piece.

The preceding detailed description of exemplary embodiments of the invention makes reference to the accompanying drawings, which show the exemplary embodiment by way of illustration. While these exemplary embodiments are described in sufficient detail to enable those skilled in the art to practice the invention, it should be understood that other embodiments may be realized and that logical and mechanical changes may be made without departing from the spirit and scope of the invention. For example, the steps recited in any of the method or process claims may be executed in any order and are not limited to the order presented. Thus, the preceding detailed description is presented for purposes of illustration only and not of limitation, and the scope of the invention is defined by the preceding description and with respect to the attached claims.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. The operations of the methods described herein may be carried out in any suitable order or simultaneously where appropriate. Additionally, individual blocks may be added or deleted from any of the methods without departing from the spirit and scope of the subject matter described herein. Aspects of any of the examples described above may be combined with aspects of any of the other examples described to form further examples without losing the effect sought.

As used herein, the terms “comprises,” “comprising,” or any other variations thereof are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “critical.”

For purposes of the instant disclosure, the term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted as a range whose lower limit is 25 and whose upper limit is 100. Additionally, it should be noted that where a range is given, every possible subrange or interval within that range is also specifically intended unless the context indicates the contrary. For example, if the specification indicates a range of 25 to 100, such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only, and decimal and fractional values (e.g., 46.7-91.3) should also be understood to be intended as possible subrange endpoints unless specifically excluded.

1. A pH-sensitive wound dressing, comprising:
a hydrogel configured for contact with a wound;
a pH-indicating dye that is incorporated within the hydrogel, wherein the pH-indicating dye demonstrates a colorimetric change within a pre-determined pH-range; and
a hydrogel covering configured to maintain contact between the hydrogel and the wound.

2. The pH-sensitive wound dressing of claim 1, wherein the hydrogel comprises a natural polymer, a synthetic polymer, or a combination thereof.

3. The pH-sensitive wound dressing of claim 2, wherein the natural polymer is selected from the group consisting of gelatin, collagen, agar-agar, hyaluronic acid, chitosan, heparin, alginate, fibrin, bacterial nanocellulose, and combinations or copolymers thereof.

4. The pH-sensitive wound dressing of claim 2, wherein the synthetic polymer is selected from the group consisting of polyvinyl alcohol, polyethylene glycol, sodium polyacrylate, polyvinylpyrrolidone, and combinations or copolymers thereof.

5. The pH-sensitive wound dressing of claim 2, wherein the hydrogel further comprises an anti-microbial agent, a dye-retention additive, or both.

6. The pH-sensitive wound dressing of claim 1, wherein the hydrogel is visually divided into sections to aid identification of a nexus of infection for the wound.

7. The pH-sensitive wound dressing of claim 1, wherein the pre-determined pH range is between about 4 and about 9.

8. The pH-sensitive wound dressing of claim 1, wherein the pH-indicating dye is selected from the group consisting of bromothymol blue, azolitmin, neutral red, methylene blue, and combinations thereof.

9. The pH-sensitive wound dressing of claim 1, wherein the hydrogel comprises a natural polymer, which is gelatin, and the pH-indicating dye is bromothymol blue.

10. The pH-sensitive wound dressing of claim 1, wherein the pH-indicating dye is configured to exhibit a first colorimetric change from yellow to blue, followed by a second colorimetric change from blue to yellow.

11. A method for monitoring a wound for infection, the method comprising the steps of:
positioning a pH-sensitive wound dressing in direct contact with the wound, wherein the pH-sensitive wound dressing comprises:
a hydrogel configured to contact the wound,
a pH-indicating dye that is incorporated within the hydrogel, wherein the pH-indicating dye demonstrates a colorimetric change within a pre-determined pH range, and
a hydrogel covering configured to maintain contact between the hydrogel and the wound;
maintaining contact between the pH-sensitive wound dressing and the wound for an observation period; and
evaluating whether the pH-sensitive wound dressing reflects the colorimetric change during the observation period.

12. The method of claim 11, wherein the observation period ranges from one second to 100 hours after the initial contact of the wound with the pH-sensitive wound dressing.

13. The method of claim 11, wherein the step of evaluating whether the pH-sensitive wound dressing reflects a colorimetric change comprises the step of checking the pH-sensitive wound dressing for the colorimetric change at a pre-determined observation interval.

14. The method of claim 11, wherein the step of evaluating whether the pH-sensitive wound dressing reflects the colorimetric change is performed by visual inspection or using a colorimeter.

15. The method of claim 14, wherein the step of evaluating whether the pH-sensitive wound dressing reflects the colorimetric change is performed by comparing the color of the hydrogel to a reference color scale by visual inspection, wherein the reference color scale depicts colors that are expected from the pH-indicating dye for pre-specified pH values within the pre-determined pH-range.

16. The method of claim 11, further comprising the step of observing the pH-sensitive wound dressing for a second colorimetric change after the colorimetric change is detected.

17. A method for preparing a pH-sensitive wound dressing, comprising the steps of:
adsorbing a pH-indicating dye within a hydrogel, wherein the pH-indicating dye demonstrates a colorimetric change within a pre-determined pH range;
positioning the hydrogel in direct contact with a wound; and
placing a hydrogel covering over the hydrogel, wherein the hydrogel covering is configured to maintain contact between the hydrogel and the wound.

18. The method of claim 17, wherein the step of adsorbing the pH-indicating dye comprises the steps of:
mixing the pH-indicating dye with a hydrogel solution; and
allowing the hydrogel solution to solidify to form the hydrogel.

19. The method of claim 17, wherein the step of adsorbing the pH-indicating dye comprises the steps of:
allowing a hydrogel solution to solidify to form the hydrogel;
covering the hydrogel with a layer of the pH-indicating dye; and
permitting the hydrogel to adsorb the pH-indicating dye for a predetermined adsorption period.

20. The method of claim 19, wherein the step of allowing the hydrogel solution to solidify comprises the step of incubating the hydrogel solution at room temperature.
